(19) United States
(12) Patent Application Publication
(10) Pub. No.: US 2026/0000320 A1
(43) Pub. Date: Jan. 1, 2026

(54) MEASUREMENT ANALYSIS

(71) Applicant: WearOptimo Pty Ltd, South Brisbane, Queensland (AU)

(72) Inventors: Anthony Mark BREWER, South Brisbane (AU); Matthew James ABURN, South Brisbane (AU); Farrukh MATEEN, South Brisbane (AU); Jeremy Gwyn Upham JONES, South Brisbane (AU)

(21) Appl. No.: 18/838,820

(22) PCT Filed: Feb. 22, 2023
(86) PCT No.: PCT/AU2023/050123

§ 371 Date: Aug. 15, 2024

(30) Foreign Application Priority Data
Feb. 24, 2022 (AU) 2022900429

(51) Int. Cl. A61B 5/145; A61B 5/00; A61B 5/1473; A61B 5/263

(52) U.S. Cl. CPC A61B 5/1451; A61B 5/0048; A61B 5/14542; A61B 5/14546; A61B 5/14735; A61B 5/263; A61B 5/6833; A61B 5/685; A61B 5/7275; A61B 2560/0242; A61B 2560/0468; A61B 2562/0209; A61B 2562/028; A61B 2562/046; A61B 2562/227

(57) ABSTRACT

A system for analyzing measurements performed on a biological subject, the system including one or more processing devices configured to acquire subject data at least in part captured by a measurement system including at least one substrate including a plurality of microstructures configured to breach a functional barrier of the subject and a sensor operatively connected to at least one microstructure, the at least one sensor being configured to measure response signals from the at least one microstructure, and analyze the subject data using at least one model to determine an indicator at least partially indicative of a physiological state of the subject.

### BACKGROUND OF THE INVENTION

The present invention relates to a method and system for analyzing measurements performed on a biological subject, and in particular to a method and system for analyzing measurements performed using a substrate including microstructures that penetrate a functional barrier of the subject.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavor to which this specification relates.

Biological markers, such as proteins, antibodies, cells, small chemicals, hormones and nucleic acids, whose presence in excess or deficiency may indicate a diseased state, have been found in blood serum and their levels are routinely measured for research and for clinical diagnosis. Standard tests include antibody analysis for detecting infections, allergic responses, and blood-borne cancer markers (e.g. prostate specific antigen analysis for detecting prostate cancer). The biological markers may originate from many organ systems in the body but are extracted from a single compartment, the venous blood.

However, this is not suitable for all conditions as often blood does not contain key biological markers for diseases originating in solid tissues, and whilst this problem has been partially overcome by taking tissue biopsies, this is time-consuming, painful, risky, costly and can require highly-skilled personnel such as surgeons.

Another serum-rich fluid is the interstitial fluid (ISF) which fills the intercellular spaces in solid tissues and facilitates the passage of nutrients, biomarkers, and excretory products via the blood stream.

WO2005/072630 describes devices for delivering bioactive materials and other stimuli to living cells, methods of manufacture of the device and various uses of the device, including a number of medical applications. The device comprises a plurality of structures which can penetrate a body surface so as to deliver the bioactive material or stimulus to the required site. The structures are typically solid and the delivery end section of the structure is so dimensioned as to be capable of insertion into targeted cells to deliver the bioactive material or stimulus without appreciable damage to the targeted cells or specific sites therein.

WO2020/069565 describes a system for performing measurements on a biological subject, the system including: at least one substrate including a plurality of plate microstructures configured to breach a stratum corneum of the subject: at least one sensor operatively connected to at least one microstructure, the at least one sensor being configured to measure response signals from the at least one microstructure; and, one or more electronic processing devices configured to: determine measured response signals; and, at least one of: provide an output based on measured response signals: perform an analysis at least in part using the measured response signals; and, store data at least partially indicative of the measured response signals.

WO2020069564 describes a system for performing fluid level measurements on a biological subject, the system including at least one substrate including a plurality of microstructures configured to breach a stratum corneum of the subject, at least some microstructures including an electrode, a signal generator operatively connected to at least one microstructure to apply an electrical stimulatory signal to the at least one microstructure and at least one sensor operatively connected to at least one microstructure, the at least one sensor being configured to measure electrical response signals from at least one microstructure. The system also includes one or more electronic processing devices that determine measured response signals, the response signals being at least partially indicative of a bioimpedance and perform an analysis at least in part using the measured response signals to determine at least one indicator at least partially indicative of fluid levels in the subject.

### SUMMARY OF THE PRESENT INVENTION

In one broad form, an aspect of the present invention seeks to provide a system for analyzing measurements performed on a biological subject, the system including one or more processing devices configured to: acquire subject data at least in part captured by a measurement system including: at least one substrate including a plurality of microstructures configured to breach a functional barrier of the subject; and, a sensor operatively connected to at least one microstructure, the at least one sensor being configured to measure response signals from the at least one microstructure; and, analyze the subject data using at least one model to determine an indicator at least partially indicative of a physiological state of the subject.

In one embodiment the subject data includes at least one of: an identifier associated with at least one of: the subject; and, a patch including the microstructures: sensor data indicative of the measured response signals: secondary sensor data indicative of measurements performed by one or more secondary sensors: subject trait data indicative of one or more subject traits: subject parameters derived from previous measurements; and, context data indicative of at least one of: environmental parameters: environmental parameters measured by one or more environmental sensors; and, a subject context.

In one embodiment the secondary sensors include physiological sensors configured to sense one or more physiological parameters or signals.

In one embodiment the one or more processing devices are configured to: analyze the subject data to determine at least one metric; and, apply the at least one metric to the model to determine the indicator, wherein the at least one model embodies a relationship between a physiological state and the at least one metric.

In one embodiment the at least one metric includes at least one of: response signals; pre-processed response signals: values derived from the response signals: subject data: an attribute of the subject data: a feature derived from an attribute of the subject data: an attribute of the context data; and, a feature derived from an attribute of the context data.

In one embodiment the attribute is statistically derived from measured response signal values and includes at least one of: a mean: a median: an average: a variance: a skew: a kurtosis; a percentile; and, a cumulative distribution function.

In one embodiment the one or more processing devices are configured to derive the features using at least one of: changes of attributes: rates of change of attributes: deviation of attributes from reference attributes: deviations of attributes from a baseline; and, one or more feature engineering algorithms.

In one embodiment the one or more processing devices are configured to process sensor data at least in part using at least one of: blind source separation algorithms: independent component analysis; and, principal component analysis.

In one embodiment the indicator is at least one of: a predictive indicator: a classification: an index value; and, a measurement value.

In one embodiment the one or more processing devices are configured to at least one of: record the indicator: generate an output including at least one of: a representation of the indicator; and, a recommendation based on the indicator; and, cause an intervention to be performed based on the indicator.

In one embodiment the one or more processing devices are configured to process the subject data by performing at least one of: anomaly detection: data cleaning: bias correction; windowing: normalization: standardization: base lining; and, signal processing.

In one embodiment the one or more processing devices are configured to: perform anomaly detection by analyzing the subject data to identify at least one of: sudden changes in response signal values: outlier response signal changes: outlier response signal values; and, changes in response signal values corresponding to events; and, at least one of: perform data cleaning by at least one of: excluding anomalies from subsequent analysis: excluding sensor data including anomalies; and, performing anomaly correction; and, use pattern matching of anomalies to identify measurement device issues: analyze the anomalies; and, determine at least one metric using the anomaly.

In one embodiment the system includes one or more secondary sensors, and wherein the one or more processing devices are configured to at least one of: synchronize the sensor data and secondary sensor data; and, process the sensor data in accordance with secondary sensor data to at least one of: perform bias correction: identify events; and, perform anomaly detection.

In one embodiment the one or more processing devices are configured to perform bias correction based on at least one of: individual sensor characteristics: environmental parameters; and, physiological parameters.

In one embodiment the one or more processing devices are configured to: segment the subject data into a number of windows; and, analyze the subject data using the windows.

In one embodiment the one or more processing devices are configured to analyze the data by analyzing at least one of: at least one metric for each window; a plurality of metrics for each window: a plurality of metrics for each of a plurality of windows; inter-window metric changes; and, intra-window metric changes.

In one embodiment the one or more processing devices are configured to normalize subject data for each window.

In one embodiment the one or more processing devices are configured to segment the subject data based on at least one of: fixed time intervals: events; events detected using secondary sensors; and, identified anomalies.

In one embodiment the one or more processing devices are configured to: generate an indicator for each window; and, generate an indicator using metrics from multiple windows.

In one embodiment the one or more processing devices are configured to standardize the subject data to establish a baseline, the standardization being performed based on at least one of: historical subject data; and, physiological parameters.

In one embodiment the one or more processing devices are configured to perform baselining by using a baseline to at least one of: adjust subject data: flag subject data of interest.

In one embodiment the one or more processing devices are configured to perform baselining by at least one of: comparison to a baseline; and, using a baselining computational model.

In one embodiment the at least one model is at least one of: a biophysical model: a computational model; a statistical model; and, a biochemical model.

In one embodiment the at least one model is obtained using reference metrics derived from subject data measured for one or more reference subjects having known physiological states.

In one embodiment the at least one model is obtained and/or fit using at least one of machine learning and statistical inference.

In one embodiment the model is obtained and/or fit using at least one of: linear or non-linear regression: logistic regression: clustering algorithms: neural networks: random forests: decision trees: Bayesian algorithms: Random effects, fixed effects or mixed effects modelling: Random field modelling: gaussian processes; and, ensemble methods.

In one embodiment the one or more electronic devices are configured to determine an indicator by performing at least one of: pattern matching: a longitudinal analysis; and, comparison to a threshold.

In one embodiment the one or more processing devices are configured to determine a physiological state indicator indicative of at least one of: a predicted physiological state of the subject: a presence, absence or degree of a medical condition: a prognosis associated with a medical condition: a presence, absence, level or concentration of a biomarker: a presence, absence, level or concentration of an analyte: fluid levels in the subject: blood oxygenation; and, bioelectric activity.

In one embodiment the measurement system includes a signal generator operatively connected to at least one microstructure to apply a stimulatory signal to the at least one microstructure.

In one embodiment the response signals or stimulatory signals are at least one of: mechanical: magnetic: thermal: electrical: electromagnetic; and, optical.

In one embodiment the measurement system includes: a patch including the substrate and microstructures; and, a monitoring device that is configured to at least one of: perform the measurements: generate the subject data: provide the subject data to the one or more processing devices; and, display an output based on the indicator.

In one embodiment the monitoring device is at least one of: inductively coupled to the patch: attached to the patch; and, placed in contact with the patch at least one of: when measurements are to be performed; and, when sensor data is retrieved from the sensor.

In one embodiment the sensor is at least one of: mounted on the patch; and, provided in the monitoring device.

In one embodiment the system includes one or more secondary sensors, and wherein the secondary sensors are at least one of: mounted on a patch: provided in the monitoring device; and, in communication with the monitoring device.

In one embodiment at least some of the microstructures include at least one electrode that at least one of: extends over a length of a distal portion of the microstructure: extends over a length of a portion of the microstructure spaced from the tip: is positioned proximate a distal end of the microstructure: is positioned proximate a tip of the microstructure: extends over at least 25% of a length of the microstructure: extends over less than 50% of a length of the microstructure: extends over about 60 μm of the microstructure; and, is configured to be positioned in a viable epidermis of the subject in use.

In one embodiment the substrate includes electrical connections to allow electrical signals to be applied to and/or received from respective microstructures.

In one embodiment at least some of the microstructures include an insulating layer extending over at least one of: part of a surface of the microstructure: a proximal end of the microstructure: at least half of a length of the microstructure: about 90 μm of a proximal end of the microstructure; and, at least part of a tip portion of the microstructure.

In one embodiment the microstructures include at least one of: plate microstructures: at least partially tapered plate microstructures: plate microstructures having a substantially rounded rectangular cross sectional shape: spaced apart substantially parallel plate microstructures: spaced apart rows of microstructures: pairs of spaced apart microstructures; and, groups of microstructures.

In one embodiment at least one of: at least some microstructures are angularly offset: at least some microstructures are orthogonally arranged: adjacent pairs of microstructures are orthogonally arranged: adjacent pairs of microstructures are angularly offset: pairs of microstructures are arranged in rows, and the pairs of microstructures in one row are orthogonally arranged relative to pairs of microstructures in other rows; and, pairs of microstructures are arranged in rows, and the pairs of microstructures in one row are angularly offset relative to pairs of microstructures in other rows.

In one embodiment the microstructures have a spacing that is at least one of: less than 1 mm: about 0.5 mm; about 0.2 mm: about 0.1 mm; and, more than 10 μm.

In one embodiment at least some of the microstructures have at least one of: a length that is at least one of: less than 300 μm: about 150 μm: greater than 100 μm; and, greater than 50 μm: a maximum width that is at least one of: greater than the length: about the same as the length: less than 300 μm: about 150 μm; and, greater than 50 μm; and, a thickness that is at least one of: less than 50 μm: about 25 μm; and, greater than 10 μm.

In one embodiment at least some of the microstructures have a tip that at least one of: has a length that is at least one of: less than 50% of a length of the microstructure: at least 10% of a length of the microstructure; and, about 30% of a length of the microstructure; and, has a sharpness of at least one of: at least 0.1 μm: less than 5 μm; and, about 1 μm.

In one embodiment the microstructures have a density that is at least one of: less than 5000 per cm²: greater than 100 per cm²; and, about 600 per cm².

In one embodiment at least some microstructures include an electrode having a surface area of at least one of: less than 200,000 μm²: about 22,500 μm²; and, at least 2,000 μm².

In one embodiment the microstructures include anchor microstructures used to anchor the substrate to the subject and wherein the anchor microstructures at least one of: include anchoring structures: have a length greater than that of other microstructures; and, enter the dermis.

In one embodiment the microstructures include a material including at least one of: a bioactive material: a reagent for reacting with analytes in the subject: a binding agent for binding with analytes of interest: a probe for selectively targeting analytes of interest: a material to reduce biofouling: a material to attract at least one substance to the microstructures: a material to repel at least one substance from the microstructures: a material to attract at least some analytes to the projections; and, a material to repel at least some analytes from the projections.

In one embodiment at least some of the microstructures are coated with a coating that at least one of: modifies surface properties to at least one of: increase hydrophilicity: increase hydrophobicity; and, minimize biofouling: attracts at least one substance to the microstructures: repels at least one substance from the microstructures; acts as a barrier to preclude at least one substance from the microstructures; and, includes at least one of: polyethylene: polyethylene glycol: polyethylene oxide: zwitterions: peptides: hydrogels; and, SAMs.

In one broad form, an aspect of the present invention seeks to provide a method for analyzing measurements performed on a biological subject, the method including, in one or more processing devices: acquiring subject data at least in part captured by a measurement system including: at least one substrate including a plurality of microstructures configured to breach a functional barrier of the subject; and, a sensor operatively connected to at least one microstructure, the at least one sensor being configured to measure response signals from the at least one microstructure; and, analyzing the subject data using at least one model to determine an indicator at least partially indicative of a physiological state of the subject.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:—

FIG. 1A is a schematic diagram of an example of a system for performing measurements on a biological subject:

FIG. 1B is a schematic underside view of an example of a patch for the system of FIG. 1A;

FIG. 2 is a flow chart of an example of a method for analyzing measurements performed using the arrangement of FIG. 1A:

FIG. 3 is a schematic side view of a further example of a system for performing measurements on a biological subject:

FIG. 4 is a schematic diagram of an example of a distributed computer architecture:

FIG. 5 is a schematic diagram of an example of a processing system:

FIG. 6 is a schematic diagram of an example of a client device:

FIGS. 7A to 7D are a flow chart of an example of a method for analyzing measurements performed using the arrangement of FIGS. 3 to 6;

FIG. 8 is a flow chart of an example of a method for training a computational model:

FIG. 9A is a schematic side view of an example of a plate microstructure:

FIG. 9B is a schematic front view of the microstructure of FIG. 9A:

FIG. 9C is a schematic underside view of an example of a patch including the microstructure of FIG. 9A:

FIG. 9D is a schematic perspective topside view of an example of substrate including pairs of blade microstructures of FIGS. 9A and 9B:

FIG. 9E is a schematic plan view of a grid of pairs of microstructures including electrical connections:

FIG. 9F is an image of an example of a patch including arrays of pairs of angularly offset plate microstructures:

FIG. 9G is a schematic side view of a specific example of a plate microstructure:

FIG. 9H is a schematic perspective view of the plate microstructure of FIG. 5G;

FIG. 9I is a schematic side view of an example of a pair of microstructures inserted into a subject for epidermal measurement:

FIG. 9J is a schematic side view of an example of a pair of microstructures inserted into a subject for dermal measurement:

FIG. 10 is a schematic diagram of an example of a basic biophysical model of living tissue:

FIG. 11A is a schematic diagram of an example of a patch in contact with saline solution for testing:

FIGS. 11B to 11D are schematic diagrams of example biophysical models for the testing arrangement of FIG. 11A:

FIG. 12A is a schematic diagram of an example of a dual channel sensing arrangement:

FIG. 12B is a schematic of an example biophysical model for a single channel of the arrangement of FIG. 12A.

FIG. 12C is a schematic of an example biophysical model for the dual channel sensing arrangement of FIG. 12A.

FIG. 13A is a schematic diagram of an example of a patch positioned in the skin for performing a bioimpedance measurement:

FIGS. 13B and 13C are schematic diagrams of example biophysical models for the bioimpedance measurement arrangement of FIG. 13A.

### DETAILED DESCRIPTION OF THE PREFERRED EMBODIMENTS

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The terms “about” and “approximately” are used herein to refer to conditions (e.g. amounts, levels, concentrations, time, etc.) that vary by as much as 20% (i.e. +20%), especially by as much as 10%, 9%, 8%, 7%, 6%, 5%, 4%, 3%, 2% or 1% to a specified condition.

As used herein, the term “analyte” refers to a naturally occurring and/or synthetic element, compound or substance, which is a marker of a condition (e.g., drug abuse), disease state (e.g., infectious diseases), disorder (e.g., neurological disorders), or a normal or pathologic process that occurs in a subject (e.g., drug metabolism), or an element, compound or substance which can be used to monitor levels of an administered or ingested substance in the subject, such as a medicament (substance that treats, prevents and/or alleviates the symptoms of a disease, disorder or condition, e.g., drug, vaccine etc.), an illicit substance (e.g. illicit drug), a non-illicit substance of abuse (e.g. alcohol or prescription drug taken for non-medical reasons), a poison or toxin (including an environmental contaminant), a chemical warfare agent (e.g. nerve agent, and the like) or a metabolite thereof. The term “analyte” can refer to any substance, including chemical and/or biological agents that can be measured in an analytical procedure, including nucleic acids, proteins, illicit drugs, explosives, toxins, pharmaceuticals, carcinogens, poisons, allergens, and infectious agents, which can be measured in an analytical procedure. The analyte may be a compound found directly in a sample such as biological tissue, including body fluids (e.g. interstitial fluid), from a subject, especially in the dermis and/or epidermis. In particular embodiments, the analyte is a compound found in the interstitial fluid. In some embodiments, the analyte is a compound with a molecular weight in the range of from about 30 Da to about 100 kDa, especially about 50 Da to about 40 kDa. Other suitable analytes are as described herein. The analyte could also include fluids within the body, such as the volumes, concentrations, presence or absence of fluids within the body. For example, the volumes or concentrations of Interstitial fluid (ISF), Intracellular fluid (ICF), or the like, could be indicative of a hydration status.

As used herein, the term “and/or” refers to and encompasses any and all possible combinations of one or more of the associated listed items, as well as the lack of combinations when interpreted in the alternative (or).

As used herein, the term “aptamer” refers to a single-stranded oligonucleotide (e.g. DNA or RNA) that binds to a specific target molecule, such as an analyte. An aptamer may be of any size suitable for binding such target molecule, such as from about 10 to about 200 nucleotides in length, especially from about 30 to about 100 nucleotides in length.

The term “bind” and variations such as “binding” are used herein to refer to an interaction between two substances, such as an analyte and an aptamer or an analyte and a molecularly imprinted polymer. The interaction may be a covalent or non-covalent interaction, particularly a non-covalent interaction.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “plurality” is used herein to refer to more than one, such as 2 to 1×10¹⁵ (or any integer therebetween) and upwards, including 2, 10, 100, 1000, 10000, 1×10⁶, 1×10⁷, 1×10⁸, 1×10⁹, 1×10¹⁰, 1×10¹¹, 1×10¹², 1×10¹³, 1×10¹⁴, 1×10¹⁵, etc. (and all integers therebetween).

As used herein, the term “predetermined threshold” refers to a value, above or below which indicates the likely or possible presence, absence or progression of a disease, disorder or condition: the presence or absence of an illicit substance or non-illicit substance of abuse: or the presence or absence of a chemical warfare agent, poison and/or toxin. For example, for the purposes of the present invention, a predetermined threshold may represent the level or concentration of a particular analyte. For example, this could be established from a corresponding sample from an appropriate control subject, such as a healthy subject, pooled samples from multiple control subjects or medians or averages of multiple control subjects, or a baseline value previously established for the subject. Thus, a level, concentration or rate of change thereof, above or below the threshold can indicate the presence, absence or progression of a disease, disorder or condition: the presence or absence of an illicit substance or non-illicit substance of abuse: or the presence or absence of a chemical warfare agent, poison and/or toxin, as taught herein. In other examples, a predetermined threshold may represent a value larger or smaller than the level or ratio determined for a control subject so as to incorporate a further degree of confidence that a level, rate of change, or ratio above or below the predetermined threshold is indicative of the presence, absence or progression of a disease, disorder or condition: the presence or absence of an illicit substance or non-illicit substance of abuse: or the presence or absence of a chemical warfare agent, poison and/or toxin. Those skilled in the art can readily determine an appropriate predetermined threshold based on analysis of samples from appropriate control subjects.

The terms “selective” and “selectivity” as used herein refer to molecularly imprinted polymers or aptamers that bind an analyte of interest without displaying substantial binding of one or more other analytes. Accordingly, a molecularly imprinted polymer or aptamers that is selective for an analyte, such as troponin or a subunit thereof, exhibits selectivity of greater than about 2-fold, 5-fold, 10-fold, 20-fold, 50-fold, 100-fold or greater than about 500-fold with respect to binding of one or more other analytes.

The term “subject” as used herein refers to a vertebrate subject, particularly a mammalian subject, for whom monitoring and/or diagnosis of a disease, disorder or condition is desired. Suitable subjects include, but are not limited to, primates: avians (birds): livestock animals such as sheep, cows, horses, deer, donkeys and pigs: laboratory test animals such as rabbits, mice, rats, guinea pigs and hamsters: companion animals such as cats and dogs: bats and captive wild animals such as foxes, deer and dingoes. In particular, the subject is a human.

System for Interacting with a Subject

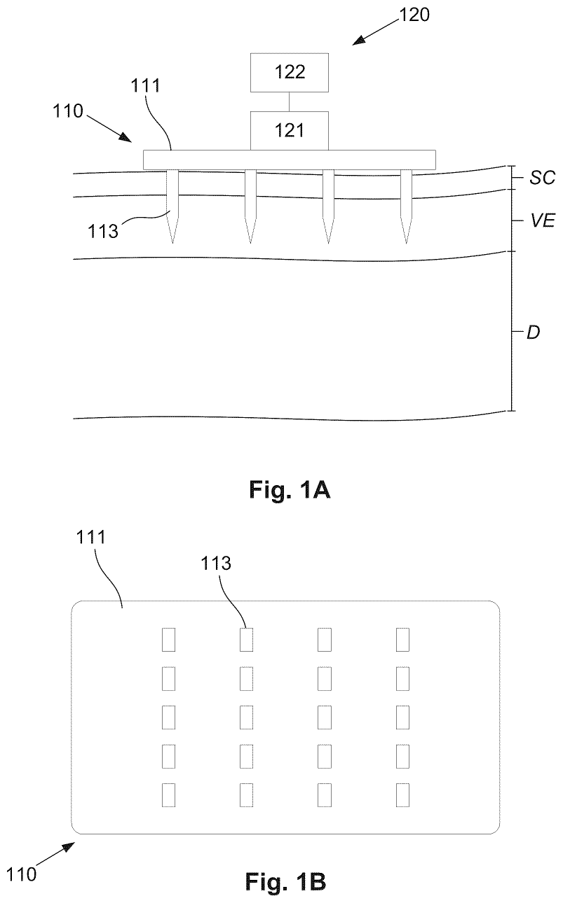

An example of a system for performing measurements on a biological subject will now be described with reference to FIGS. 1A and 1B.

In this example, the system includes at least one substrate 111 having one or more microstructures 113. In use, the microstructures are configured to breach a functional barrier associated with a subject. In the current example, the functional barrier is the stratum corneum SC, and the microstructures are configured to breach the stratum corneum SC by penetrating the stratum corneum SC and entering at least the viable epidermis VE. In one particular example, the microstructures are configured to not penetrate a boundary between the viable epidermis VE and the dermis I), although this is not essential and structures that penetrate into the dermis could be used as will be described in more detail below.

Whilst this example is described with respect to breaching of the stratum corneum SC, it will be appreciated that this is not essential, and the techniques could equally be applied to other functional barriers. In this regard, a functional barrier will be understood to include any structure, boundary, or feature, whether physical or otherwise, that inhibits the passage of signals, and/or analytes, such as biomarkers. For example, functional barriers could include one or more layers, a mechanical discontinuity, such as a discrete change in tissue mechanical properties, a tissue discontinuity, a cellular discontinuity, a neural barrier, a sensor barrier, a cellular layer, skin layers, mucosal layers, internal or external barriers, an inner barrier within an organ, an outer barrier of organs other than the skin, epithelial layers or endothelial layers, or the like. Functional barriers could also include other internal layers or boundaries, including optical barriers such as a melanin layer, electrical barriers, molecular weight barriers that prevent passage of a biomarkers with certain molecular weights, a basal layer boundary between the viable epidermis and dermis, or the like.

The nature of the microstructure will vary depending upon the preferred implementation. In one example, the microstructures could include needles, but this is not essential and more typically structures, such as plates, blades, or the like, are used, as will be described in more detail below.

The substrate and microstructures could be manufactured from any suitable material, and the material used may depend on the intended application, for example depending on whether there is a requirement for the structures to be optically and/or electrically conductive, or the like. The substrate can form part of a patch 110, which can be applied to a subject, although other arrangements could be used for example, having the substrate form part of a housing containing other components.

In one example at least one sensor 121 is provided, which is operatively connected to at least one microstructure 113, thereby allowing response signals to be measured from respective microstructures 113. In this regard, the term response signal will be understood to encompass signals that are intrinsic within the subject, such ECG (Electrocardiogramals, or the like, or signals that are induced as a result of the application of stimulation, such as bioimpedance signals, or the like.

The nature of the sensor will vary depending on the preferred implementation and the nature of the sensing being performed. For example, the sensing could include sensing electrical signals, in which case the sensor could be a voltage or current sensor, or the like. Alternatively, optical signals could be sensed, in which case the sensor could be an optical sensor, such as a photodiode, CCD (Charge Coupled Device) array, or similar, whilst temperature signals could be sensed using a thermistor or the like.

The manner in which the sensor 121 is connected to the microstructure(s) 113 will also vary depending on the preferred implementation. In one example, this is achieved using connections between the microstructure(s) 113 and the sensor, with the nature of the connections varying depending upon the signals being sensed, so that the connections could include electrically conductive elements to conduct electrical signals, a wave guide, optical fiber or other conductor to conduct electromagnetic signals, or thermal conductor to conduct thermals signals. Connections could also include wireless connections, allowing the sensor to be located remotely. Ionic connections could also be used. Furthermore, connections could be provided as discrete elements, although in other examples, the substrate provides the connection, for example, if the substrate is made from a conductive plate which is then electrically connected to some or all of the microstructures. As a further alternative, the sensor could be embedded within or formed from part of the microstructure, in which case connections between sensor and microstructures may not be required. Alternatively, microstructures could be imprinted directly onto a substrate, such as a flexible printed circuit board, or similar.

The sensor 121 can be operatively connected to all of the microstructures 113, with connections being collective and/or independent. For example, one or more sensors could be connected to different microstructures to allow different measured response signals to be measured from different groups of microstructures 113. However, this is not essential, and any suitable arrangement could be used.

In addition to providing sensing, in some examples, the microstructures 113 could additionally and/or alternatively be configured to provide stimulation. For example, microstructures could be coupled to a signal generator that generates a stimulatory signal, as will be described in more detail below. Such stimulation could again include electrical stimulation, using a voltage or current source, optical stimulation, using a visible or non-visible radiation source, such as an LED or laser, thermal stimulation, mechanical stimulation, such as vibration, or the like, and could be delivered via the same microstructures used for measuring response signals, or different microstructures, depending on the preferred implementation. Additionally and/or alternatively, stimulation could be achieved using other techniques, such as through exposure of the subject to the microstructures and materials thereon or therein. For example, coatings can be applied to the microstructures, allowing material to be delivered into the subject beyond the barrier, thereby stimulating a response within the subject.

These options allow a range of different types of sensing to be performed, including detecting electrical signals within the body, such as ECG signals, plethysmographic signals, electromagnetic signals, or electrical potentials generated by muscles, neural tissue, blood, or the like, detecting photoplethysmographic effects, electromagnetic effects, such as fluorescence, detecting mechanical properties, such as stress or strain, or the like. Sensing could include detecting the body's response to applied electrical signals, for example to measure bioimpedance, bioconductance, or biocapacitance, detecting the presence, absence, level or concentration of analytes, for example by detecting electrical or optical properties, or the like.

For example, measurements can be performed by passing a current between electrodes, with measurements of the resulting signal between the electrodes being used to detect changes in the electrical properties and hence, the presence, absence, level or concentration of analytes. In this regard, the electrical output signal can be indicative of any one or more of a voltage, a current, a resistance, a capacitance, a conductance, or an impedance, or a change in any of these variables. Thus, signals could be potentiometric, amperometric, voltametric, impedimetric, or the like.

For example, impedance measurements, such as in electrochemical impedance spectroscopy (EIS), investigate the dynamics of the bound analyte or the charge transfer in the bulk or the interfacial region of the MIP and/or aptamer. In this regard, when an MIP (especially a conductive MIP) captures a target analyte, the MIP cavities are filled, hindering the diffusion of ions in the bulk polymer. In addition, captured analyte can strain the structure of the conductive MIP causing increase in the charge transfer in the polymer. Similarly when an aptamer captures a target analyte, the captured analyte can change the structure of the aptamer changing the electrical properties. The measurement only requires ions in the samples and can be done without a redox moiety.

In this example, the electrodes can be arranged in pairs, although alternatively the system could measure impedances between different groups of electrodes, for example with one group acting as a working electrode and the other group working as a counter electrode.

In a further example, voltametric/amperometric techniques can be used, including cyclic voltammetry (CV), linear sweep voltammetry (LSV), differential pulse voltammetry (DPV), square wave voltammetry (SWV), and chronoamperometry (CA).

In this example, a current output is generated from the redox reaction of the electroactive species (redox moiety) which takes place on the conductive material (e.g gold microstructures). When analyte of interest is captured in the MIP (especially insulating MIP coating), the MIP cavities are filled thereby blocking/hindering the diffusion of the redox moieties towards the gold surface. Decrease in the penetration of the analyte results in a decrease in the current output. Similarly, when an analyte of interest is captured in the aptamer, the structure of the aptamer changes resulting in the redox moieties moving relative to the microstructure surface, thereby altering the current output.

Since a redox reaction is required in this type of transduction, some researchers incorporate a redox moiety in the polymeric matrix.

In this example, reference electrodes might also be provided, in which case electrodes might be arranged in three groups, including working, counter and reference electrodes. The reference electrodes need only be in the vicinity of the working and counter electrodes, so that, for example, electrodes could be arranged in pairs of working and counter electrodes, with a row of pairs of electrodes being used as reference electrodes.

In a further example potentiometric measurements can be performed in which an electrical output is generated in response to binding of target analyte in the MIP and/or aptamer. Here the change in the voltage corresponding to the amount of analyte bound in the MIP and/or aptamer is measured. Potentiometric techniques can be found in sensor like ion selective electrodes (ISE) and field-effect transistors (FET).

Other measurement techniques include mass sensitive acoustic transducers such as surface-acoustic wave (SAW) oscillator, Love-wave oscillator, or quartz crystal microbalance. (QCM). In binding of analyte could be quantified via the change in the oscillation frequency resulting from the mass change at the oscillator surface.

The system further includes one or more electronic processing devices 122, which can form part of a monitoring device, and/or could include electronic processing devices forming part of one or more processing systems, such as computer systems, servers, client devices, or the like as will be described in more detail below. In use, the processing devices 122 are adapted to receive signals from the sensor 121 and either store or process the signals. For ease of illustration the remaining description will refer generally to a processing device, but it will be appreciated that multiple processing devices could be used, with processing distributed between the devices as needed, and that reference to the singular encompasses the plural arrangement and vice versa.

### Measurement Analysis

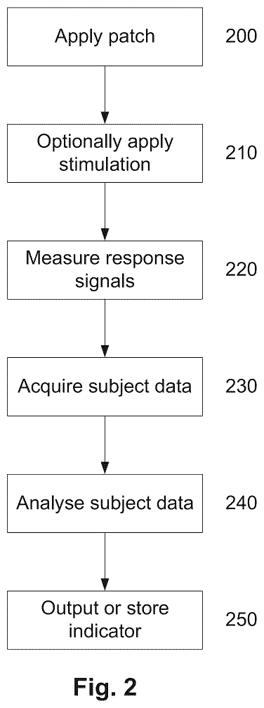

An example of the manner in which measurements are processed and analyzed will now be described with reference to FIG. 2.

In particular, in this example, at step 200 the patch is applied to the subject so that one or more microstructures breach, and in one example, penetrate the functional barrier. For example, when applied to skin, the microstructures could penetrate the stratum corneum and enter the viable epidermis as shown in FIG. 1A. This could be achieved manually and/or through the use of an actuator, to help ensure successful penetration.

At step 210 stimulation signals can optionally be applied to one or more of the microstructures, depending on the nature of the measurement process being performed. Response signals within the subject are measured at step 220, with subject data indicative of the measured response signals being provided to the electronic processing device 122 at step 230.

It will be appreciated that the electronic processing device 122 could acquire at least some subject data directly from the sensor 121, although this is not essential and in other examples, the subject data could be transferred via an intervening device, such as a monitoring device and/or client device, as will be described in more detail below. Similarly, the subject data could be stored in a database or similar, and retrieved as required, depending on the preferred implementation. The subject data could include the raw response signals, and/or could be derived from the response signals, for example using signals processing techniques, analysis, or the like. For example, the subject data could include a digitized version of the response signals, or could include values of parameters extracted from the response signals, such as maximum or minimum values, or the like. The subject data may also include additional information, such as an identifier to identify a source of the data, the identity of the subject or the like, and may also include data from other sources, including secondary data and context data, as will be described in more detail below.

The processing device 122 then analyzes the subject data using a model at step 240, using results of the analysis to generate a physiological state indicator, which can then be stored and/or output at step 250.

The subject data can be analyzed in any suitable manner depending on the preferred implementation, and the nature of subject data. For example, this can involve generating one or more metrics, which are then applied to a model, such as a biophysical model, a computational model, a statistical model and/or a biochemical model, allowing the indicator to be generated. Generation of metrics can involve an analysis of the subject data, where the nature of the analysis and the metrics generated may vary depending on the preferred implementation and/or the nature of the measurements performed. For example, the analysis could include statistical analysis, which is used to generate statistical attributes, with the metrics including or being derived from the statistical attributes. Thus, data could be analyzed to determine attributes such as a mean or median response signal value, a change in response signal value, or the like, with features being derived such as changes in mean values over time. Different combinations of attributes and/or features could then be used to form the metrics.

Whilst the analysis might be performed on raw values of the response signals, in other examples, additional analysis could be performed. For example, when performing impedance measurements, raw voltage measurements could be combined with information regarding applied current signals to derive impedance values, with the metrics being derived from the impedance values.

Additionally and/or alternatively, analysis might not be required in the generation of metrics, with subject data, potentially including raw response signals, pre-processed response signals, values derived from response signals, or the like, being applied directly to the models, so that the signals themselves act as the metrics.

The models are typically at least partially indicative of a relationship between different physiological states and the values of the one or more metrics, and/or subject data/raw response signals, and could be of any appropriate form. For example, the model could be any one or more of a biophysical model, a computational model, a statistical model and/or a biochemical model.

In this regard, a biophysical model is typically a system of equations or quantitative relations that relates biological, chemical or physical properties or states of a biological system to each other, and/or to observations or measurements of the system, and/or describes the evolution of those states and relations over time. One example is an equivalent circuit model of biological tissue impedance such as the Cole-Cole model. A second example is an equation describing the effect of artery stiffness on blood flow. A third example is a hidden Markov model describing probabilistic transitions of a hormonal signaling system between a finite set of states dependent on bodily or environmental conditions. A fourth example is a system of stochastic differential equations describing the dynamics of the electrophysiological activity of cardiac tissues and the relation of this activity to an ECG signal measured by a device.

Such models could be derived using a variety of techniques and could include models known in the art. For example, the models can be derived from reference metrics measured for one or more reference subjects having known physiological states. For example, in the case of computational models, these can be obtained by applying machine learning and/or statistical inference to the reference metrics. Thus, it will be appreciated that in practice sets of reference subject data, similar in form to the subject data, are collected for a plurality of reference subjects for which a variety of different physiological states have been diagnosed. The collected reference subject data are used to calculate reference metrics and/or train the computational model so that the computational model can be used to assist in discriminating between different physiological states, based on the subject's data and/or metrics derived from the subject's data. The nature of the computational models will vary depending on the implementation and examples will be described in more detail below.

In one example, the model(s) are directly indicative of a physiological state, in which case the metrics can be applied to the model to determine the physiological state indicator. However, this is not essential and in another example the model(s) are used to determine information which is then used to determine the physiological state indicator.

For example, when estimating fluid levels, this could involve examining the applied stimulatory signals and values of the measured response signals, using these to calculate a bioimpedance within the epidermis, which in turn allows an indicator indicative of fluid levels to be derived. For example, the Cole-Cole model can be used to derive values of intra-cellular fluid levels, extra-cellular fluid levels, and total body water. Furthermore, it will be understood that fluids within the body, such as interstitial fluid, contain ions, such as Sodium (Na+), Potassium (K+), Calcium (Ca²+), Chloride (Cl—), Bicarbonate (HCO₃—) and Phosphate (HPO₄²—). As fluid levels increase or decrease, for example as the subject's level of hydration increases or decreases, there will be a corresponding fall or rise in ion concentrations, thereby resulting in a change in conductivity of the fluid. Accordingly, measuring the impedance of the fluid can in turn be used to derive information regarding fluid conductivity, which is in turn indicative of ion concentrations and hence fluid levels. Thus, it will be appreciated that this allows changes in impedance to be used to track changes in fluid levels and hence a hydration state of the subject. Such fluid levels could include any one or more of interstitial fluid levels, a change in interstitial fluid levels, an ion concentration in interstitial fluid, a change in an ion concentration in interstitial fluid, an ion concentration, a change in an ion concentration, a total body water, intracellular fluid levels, extracellular fluid levels, plasma water levels, fluid volumes, hydration levels, skin surface fluid levels, or the like. In any event, it will be appreciated that in this instance, the model might provide an output corresponding to an impedance or fluid level, which is then interpreted to determine a state of hydration. This could be performed heuristically, or could involve the use of a second computational model, which takes the impedance or fluid level output from the first model and optionally combines this with one or more other metrics, to output a hydration state. For example, a Cole-Cole model could be used to derive information regarding intra-cellular or extra-cellular fluid levels, with a computational model being used to track changes in these fluid levels, including rates of change, to calculate a hydration indicator indicative of a subject's hydration state. Alternatively, a hydration state could be calculated directly from the response signal measurements. Thus, it will be appreciated that multiple models can be used in conjunction, depending on the preferred implementation, and the physiological state being determined.

It will be appreciated that as sensor data could be collected relating to a wide range of different measurements, this allows a range of different physiological states to be monitored. For example, the indicator could be indicative of the presence, absence, degree or prognosis of one or more medical conditions, a presence, absence, level, concentration or changes or rates of change in a level or concentration of a biomarker, a presence, absence, level, concentration, changes or rates of change in a level or concentration of an analyte, a presence, absence, grade, change or rates of change in a cancer, fluid levels, changes or rates of change in fluid levels in the subject, levels, changes or rates of change in levels of blood oxygenation, levels, changes or rates of change in levels of a tissue inflammation state, levels, changes or rates of change in levels of bioelectric activity, such as nerve, brain, muscle or heart activity, or a range of other health states.

In one example, the physiological state indicator can be indicative of a likelihood of the subject having a particular physiological state, for example indicating that the user has a 95% chance of being over- or under-hydrated. However, this is not necessarily essential, and it will be appreciated that the physiological state indicator could alternatively be indicative of a measured value, such as a fluid level, which is then interpreted by a clinician to determine if the subject is over- or under-hydrated. It will also be appreciated from this that the indicator could be indicative of a current physiological state of the user, but could additionally and/or alternatively be indicative of a predicted future physiological state. For example, during a physical activity, the indicator might be used to monitor both a current and future expected hydration state, enabling a user to determine the subject's current hydration, but also when the subject might be predicted to become adversely hydrated.

In any event, the above described method utilizes a model in order to analyze response signals measured using microstructures that breach a functional barrier within the subject, in order to allow an indicator indicative of a physiological state to be generated. In one particular example, this is achieved using a machine learning or other similar technique to generate a computational model, which is then used in performing the analysis. This is typically performed utilizing certain defined metrics derived from the response signals. In another example, this is achieved using a statistical inference technique to infer parameters and/or variables of a physical, biophysical, biochemical or statistical model, which is then used in performing the analysis.

As part of this, the above described system operates by providing microstructures that are configured to breach a barrier, such as the stratum corneum, allowing these to be used to measure response signals within the subject, such as within the epidermis and/or dermis. These response signals can then be processed and subsequently analyzed, allowing the indicator to be derived.

The system can be configured so that measurements are performed at a specific location within the subject, such as within the epidermis only, the dermis only, or the like. This allows targeted analyte detection to be performed with a high level of accuracy, providing higher quality data for more precise measures of analytes. Furthermore, constraining the location in which measurements are performed ensures these are repeatable, allowing for more accurate longitudinal monitoring.

In contrast to traditional approaches, breaching and/or at least partially penetrating a functional barrier, such as the stratum corneum, allows measurements to be performed from within or under the barrier, and in particular within the epidermis and/or dermis, resulting in a significant improvement in the quality and/or magnitude of response signals that are detected. In particular, this ensures that the response signals accurately reflect conditions within the human body, and in particular within the epidermis and/or dermis, such as the presence, absence, level or concentration of biomarkers, the impedance of interstitial fluid, or the like, as opposed to traditional external measurements, which may be unduly influenced by the environment outside the barrier, such as the physical properties of the skin surface, such as the skin material properties, the presence or absence of hair, sweat, mechanical movement of the applied sensor, or the like. Additionally, by penetrating the stratum corneum but not the dermis, this can allow measurements within the epidermis to be improved, for example reducing interference from fluid level changes in the dermis. However, it will be appreciated this will depend on the intended application, and in some situations, measurements from multiple sensors may be used in combination, for example, combining skin surface measurements and epidermal measurements.

For example, this allows accurate measurement of high molecular weight biomarkers to be performed, which would otherwise only pass through the skin poorly. A good example of this, is glucose, which whilst present externally, such as in sweat, is typically only present in low concentrations, and often time delayed, meaning the concentration in sweat does not necessarily reflect current glucose levels within the body. In contrast, by breaching the barrier, in this case the stratum corneum, this allows far more accurate measurements to be performed. It will be appreciated that similar considerations apply to a wide range of different biomarkers or signals, and associated barriers that otherwise prevent accurate measurement of the biomarkers or signals.

For example, in the case of impedance measurements microstructure electrodes tend to measure impedances different from those measured by surface electrodes, which is indicative of the fact that the microstructure electrodes can measure physiological signals distinct from skin surface impedance, meaning the measured impedance is more indicative of conditions within the body. As the contribution of the skin surface impedance is significant in magnitude this can result in changes in impedance within the body being masked, meaning skin surface measurements alone are less likely to be able to detect meaningful changes.

Additionally, in some examples, the microstructures only penetrate the barrier a sufficient distance to allow a measurement to be made. For example, in the case of skin, the microstructures are typically configured to enter the viable epidermis and not enter the dermal layer. This results in a number of improvements over other invasive techniques, including avoiding issues associated with penetration of the dermis, such as pain caused by exposure of nerves, erythema, petechiae, or the like. Avoiding penetrating the dermal boundary also significantly reduces the risk of infection, allowing the microstructures to remain embedded for prolonged periods of time, such as several days, which in turn can be used to perform longitudinal monitoring over a prolonged time periods. However, in some instances, such as when detecting troponin or a subunit thereof, penetration of the dermal barrier may be required.

It will be appreciated that the ability of the microstructures to remain in-situ is particularly beneficial, as this ensures that measurements are made at the same site within the subject, which reduces inherent variability arising from inaccuracies of replacement of measuring equipment which can arise using traditional techniques. Despite this, it will be appreciated that the system can be used in other manners, for example to perform single time point monitoring or the like.

In one example, this allows the arrangement to be provided as part of a wearable device, enabling measurements to be performed that are significantly better than existing surface based measurement techniques, for example by providing access to signals or biomarkers that cannot otherwise pass through the barrier, but whilst allowing measurements to be performed whilst the subject is undergoing normal activities and/or over a prolonged period of time. This in turn enables measurements to be captured that are more accurately reflective of the health or other status of the subject. For example, this allows variations in a subject's condition during a course of the day to be measured, and avoids measurements being made under artificial conditions, such as within a clinic, which are not typically indicative of the actual condition of the subject. This also allows monitoring to be performed substantially continuously, which can allow conditions to be detected as they arise, for example, in the case of myocardial infarction, cardiovascular disease, vomiting, diarrhea, dehydration or similar, which can allow more rapid intervention to be sought.

The above described system can be applied to any part of the body, and hence could be used with a wide range of different functional barriers. For example, the functional barrier could be an internal or external barrier, a skin layer, a mucosal layer, a meningeal layer, an inner barrier within an organ, an outer barrier of an organ, an epithelial layer, an endothelial layer, a melanin layer, an optical barrier, an electrical barrier, molecular weight barrier, basal layer or the stratum corneum. Thus, the microstructures could be applied to the buccal mucosa, dura mater, the eye, or another epithelial layer, endothelial layer, or the like. The following examples will focus specifically on application to the skin, with the functional barrier including some or all of the stratum corneum, but it will be appreciated that this is intended to be illustrative and is not intended to be limiting.

A number of further features will now be described.

In one example, the subject data includes an identifier associated with the subject and/or a patch including the microstructures. This allows the subject data to be uniquely associated with a subject and/or patch, so that the processing device can ensure that subject data for a respective subject is correctly analyzed.

The subject data can also include sensor data indicative of the measured response signals, and optionally secondary sensor data indicative of measurements performed by one or more secondary sensors. In this regard, the patch may include additional sensors, or separate sensors could be provided for example as part of a different monitoring device, such as a smartwatch, medical monitoring equipment, or the like, which are able to collect additional information that can assist in analyzing the sensor data and/or generating an indicator. Such secondary sensors could include physiological sensors configured to sense one or more physiological parameters, such as a subject temperature, heart rate, level of activity and/or movement or the like.

The subject data can also include subject trait data, such as details of subject traits. The traits are typically physiological traits such as the subject's age, weight, height, sex, or the like. Whilst this information could be measured by suitable sensors, as the traits typically don't vary significantly over short time frames, this might not be required, and alternatively, the subject trait data could be obtained from alternative sources, such as medical records, user input, or the like. The subject data may also include learned parameters, which could be derived using the model, for example, based on the results of previous measurements.

From this, it will be appreciated that whilst some of the subject data might be obtained from the measurement system, subject data could also be obtained from additional sources, such as other separate sensors, medical records, user input, or the like.

In addition the subject data might also include context data. In this regard, the context data is typically information regarding the context in which the measurement is performed. The context data can include environmental parameters, such as details of the weather, ambient temperature, humidity, or the like, and such environmental parameters could be measured using one or more environmental sensors and/or could be retrieved from an external source, such as a meteorological provider, or the like. Context data could also include a context in terms of activities currently and/or recently performed by the subject, such as whether the subject is currently or has recently undertaken physical activity, undergone stress, or the like.

Any one or more of the sensor data, the secondary sensor data, subject trait data and/or context data can be used to determine at least one metric, so for example, information regarding the subject's height or weight, as well as the ambient temperature could be relevant in interpreting fluid levels and understanding the impact of these on hydration status. Additionally and/or alternatively, the secondary sensor data, subject trait data and/or context data could be used to either control the measurement system and/or analysis of subject data. For example, on a hot day, it might be necessary to more closely monitor a subject's hydration, meaning it might be necessary to measure the subject's fluid levels more frequently, sample recorded measurements with a greater frequency, or alter a window used in data analysis as described in more detail below.

As previously mentioned, the metric(s) could include an attribute of the subject data and/or a feature derived from an attribute of the subject data. The attributes are typically statistically derived from measured response signal values and can include measures such as a mean, a median, an average, a variance, a skew; a kurtosis, a percentile, a cumulative distribution function, or the like. Meanwhile, the processing device can derive the features using a range of techniques, such examining changes of attributes, rates of change of attributes, deviation of attributes from reference attributes, deviations of attributes from a baseline, one or more feature engineering algorithms, or the like.

Signal processing can also be performed using a range of suitable techniques, such as using filtering techniques, Hilbert transforms, spectral analyses, time-frequency analyses, cross-correlation analyses, coherency analyses, blind source separation algorithms, independent component analysis, principal component analysis, or the like, with this optionally being performed on the sensor data prior to determination of the attributes.

In one example, the physiological state indicator could be a classification, an index value or a measurement value, although it will be appreciated that this is not essential and other suitable forms of indicator could be used, as will be described in more detail below.

Once generated, the processing device can be configured to record the indicator, for example adding the indicator to a health care record or similar, generate an output including a representation of the indicator and/or a recommendation based on the indicator, for example recommending fluids are ingested to aid rehydration, or trigger an action or intervention, such as alerting a clinician, trainer or guardian, or controlling a medical device, for example to administer medication or similar.

In one example, the processing device is configured to process the subject data by performing one or more of anomaly detection, data cleaning, bias correction, windowing, normalization, standardization, base lining, and, signal processing.

Anomalies may arise in the subject data for a number of reasons. For example, anomalies might be spurious measurements or values, arising as a result of external factors, such as movement of the patch, sensor inaccuracies, or the like. Spurious measurements may also arise as a result of internal factors not relevant to the bodily state of interest, such as nearby muscle activity, or the like. Accordingly, in one example, the processing device is configured to perform anomaly detection by analyzing the subject data to identify sudden changes in response signal values, outlier response signal changes, outlier response signal values, changes in response signal values corresponding to events, or the like. In the event that anomalies in the form of spurious artefacts are detected, the processing device can then perform data cleaning by excluding anomalies from subsequent analysis, excluding sensor data including anomalies, such as excluding entire windows of sensor data containing anomalies, or by performing anomaly correction. Removing such anomalies can avoid these skewing the subject data, which in turn can lead to the generation of inaccurate indicators.

In addition to, and/or as alternative to cleaning data, the processing device can use pattern matching of anomalies to identify measurement device issues. This could for example be used to determine that the patch has not successfully breached the functional barrier, to identify a faulty sensor, sensor drift, or the like, which can in turn be used to take corrective action, such as applying a new patch, replacing a sensor, performing bias correction, or the like.

As previously mentioned, the system can include one or more secondary sensors. In this example, the processing device is typically configured to synchronize the sensor data and secondary sensor data and/or process the sensor data in accordance with secondary sensor data. This can be used for a variety of purposes, such as performing bias correction, identifying events or performing anomaly detection. For example, if a subject undergoes significant movement while a measurement is being performed, this could lead to inaccuracies in the measurement and the resulting sensor data. In this example, using a secondary movement sensor to detect movement can be used to exclude sensor data captured during the movement. This is particularly beneficial as the sensor data might have a value that on the face of it appears reasonable, for example falling within an expected range, but is nevertheless inaccurate due to the movement that occurred.

It will also be appreciated that anomalies may also arise that are indicative of a physiological state of the user. For example, outlier values in heart rate measurements might be indicative of arrhythmia or other physiological conditions. Accordingly, alternatively some anomalies might be included in analysis to determine the physiological state, for example using the anomalies as metrics.

The processing device can be configured to perform bias correction and/or calibration based on individual sensor characteristics, environmental parameters and/or physiological parameters. For example, this might take into account the fact that values captured by individual sensors may vary. In this case, a correction can be applied, for example based on factory measurements, thereby ensuring the captured sensor data is an accurate reflection of physiological state.

In one example, the processing device can be configured to segment the subject data into a number of windows and then analyze the subject data using the windows. The use of windowing in this fashion can be used to identify segments of data captured at a similar time and/or under similar conditions, allowing this to be analyzed meaningfully. For example, fluid levels may be sufficiently different at rest, or when the subject is asleep, compared to when a user is active. Accordingly, analyzing fluid levels over a period of time corresponding to an entire day, may render statistical values, such as a mean or variance, effectively meaningless. However, by using windows of a shorter time period, this allows measurements to be analyzed more meaningfully. Windowing can be performed using a variety of different approaches, such as segmenting the subject data based on fixed time intervals, fixed amounts of recorded samples, events including those detected using secondary sensors, such as sleep onset, and/or identified anomalies. For example, windowing could be linked to physical activity, exertion, temperature, humidity, or the like, so that the window duration changes when a subject is more active and/or conditions are more adverse, as a result of it being more likely that the subject will dehydrate faster. Similarly, windowing could be linked to medication or a medical procedure, such as receiving IV fluids or diuretics etc, which can again impact on the rate at which a subject might become dehydrated.

The processing device can analyze the data by analyzing at least one metric for each window, a plurality of metrics for each window, a plurality of metrics for each of a plurality of windows, inter-window metric changes or intra-window metric changes, depending on the preferred implementation and/or the nature of the indicator being generated. Similarly, the processing device can generate an indicator for each window and/or generate an indicator using metrics from multiple windows.

The processing device can normalize subject data, with this optionally being performed for each window. Such normalization can maximize the magnitude of changes in the data, for example setting a maximum measured response signal value to 1 and a minimum value to 0, which in turn can help analyze changes in response signals more effectively.

In some examples, the processing device can standardize the subject data to establish a baseline, with this being performed based on historical subject data and/or physiological parameters. For example, a baseline of normal fluid levels could be established by monitoring the subject over a period of time or could be based on reference baselines established for a normal population having similar physiological parameters to the subject, such as a similar age, weight, height, sex, or the like. The baseline can be used to adjust subject data and/or flag subject data of interest, for example identifying measurements that deviate from the baseline, or to correct subject data, for example correcting for sensor drift by aligning measurements in accordance with the baseline. Baselining can be performed using a range of different techniques, including performing comparison to a baseline previously established for the subject, and/or using a baselining computational model, depending on the preferred implementation. For example, this process could include estimating subject-specific biological parameters from historical subject data, and using these to establish a subject specific baseline. Similarly, machine learning models, such as neural networks, could be further trained to learn individual subject characteristics.

As mentioned above, the computational model can be obtained and/or fit by applying machine learning or statistical inference to reference metrics derived from subject data measured for one or more reference subjects having known physiological states. The nature of the model, and the training performed, can be of any appropriate form and could include any one or more of decision tree learning, random forest, logistic regression, polynomial regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail.

Other models that can be used include state space models, dynamic causal models, multifidelity models, computational Bayesian inference, Markov Chain Monte Carlo, particle filters, ensemble Kalman filters, sequential Monte Carlo, variational inference, approximate Bayesian Computation, or the like. Example statistical models that may be used include linear or non-linear regression models, mixture models, Hidden Markov Models, random field models, Kalman filters, random effects models, fixed effects models, mixed effects models, hierarchical Bayesian models, or the like.

The indicators can be generated directly from model outputs, and/or could require further processing, for example by performing pattern matching, longitudinal analysis or comparison to a threshold.

As previously mentioned, the measurement system can include a signal generator operatively connected to at least one microstructure to apply a stimulatory signal to the at least one microstructure. The nature of the stimulatory signal can vary depending on the preferred implementation, and could include mechanical, electromagnetic, thermal, or electrical stimulation. The stimulatory signal could be used to allow the response signal to be measured and/or could be used to trigger a biological response, which is then measured. For example this can be used to cause electroporation, to induce local mediators of inflammation, which can in turn release biomarkers, allowing levels or concentrations of these to be measured. In this regard, electroporation, or electropermeabilization, involves applying an electrical field to cells in order to increase the permeability of the cell membrane, allowing chemicals, drugs, RNA, or DNA to be introduced into the cell. In another example, stimulation can be used to disrupt a boundary within the subject, for example disrupting a dermal boundary allowing biomarkers within the dermal layer to be detected in the viable epidermis, without requiring penetration of the dermal layer by the microstructures. In a further example, stimulation can be used to trigger additional effects. So for example, an electrical or mechanical signal could be used to disrupt a coating on the microstructures, causing material to be released, which can in turn induce a chemical or other stimulation.

Stimulatory signals could also be applied to the microstructures to alter the microstructure form or function. For example, polymer microstructures could be induced to grow or shrink along their length or width with an applied electric field or temperature, whilst microstructures could be configured to move between a retracted flat position and an extended upright position, in order to penetrate and then retract from the skin or other barrier.

The measurement system typically includes a patch including the substrate and microstructures and a separate monitoring device that is configured to perform the measurements, and generate at least some of the subject data, provide at least some of the subject data to the one or more processing devices and/or display an output based on the indicator. The nature of the monitoring device will vary depending on the preferred implementation and whilst this could be fixed to the patch, alternatively, this could be a physically separate device that communicates with the patch as needed, for example when measurements are to be performed and/or when sensor data is retrieved from the sensor. Such communication could be achieved via wired or wireless connections, and in one example is performed inductively allowing the patch to be effectively powered by the monitoring device, thereby reducing patch complexity.

It will be appreciated from this that the sensor could be mounted on the patch and/or provided in the monitoring device, and that similarly the secondary sensors could also be mounted on the patch, provided in the monitoring device or client device, or otherwise in communication with the monitoring device or client device.

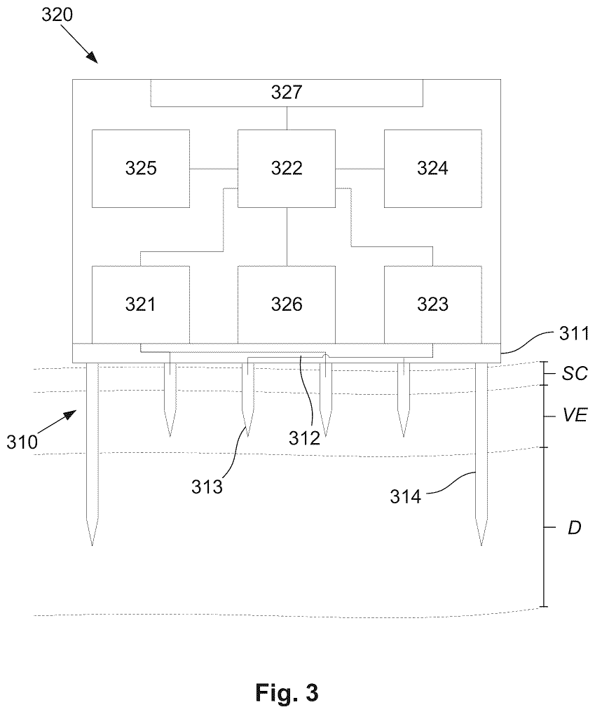

A specific example of a system for performing measurements in the biological subject will now be described with reference to FIGS. 3 to 6.

In this example, the system includes a monitoring device 320, including a sensor 321 and one or more electronic processing devices 322. The system further includes a signal generator 323, a memory 324, an external interface 325, such as a wireless transceiver, an actuator 326, and an input/output device 327, such as a touchscreen or display and input buttons, connected to the electronic processing device 322. The components are typically provided in a self-contained housing.

The nature of the signal generator 323 and sensor 321 will depend on the measurements being performed, and could include a current source and voltage sensor, laser or other electromagnetic radiation source, such as an LED, and a photodiode or CCD sensor, or the like. The monitoring device might include an actuator 326, such as a spring or electromagnetic actuator in combination with a piezoelectric actuator or vibratory motor coupled to the housing, to bias and vibrate the substrate relative to an underside of the housing, to thereby urge the microstructures into the skin, whilst the transceiver is typically a short-range wireless transceiver, such as a Bluetooth system on a chip (SoC).

The processing device 322 executes software instructions stored in the memory 324 to allow various processes to be performed, including controlling the signal generator 323, receiving and interpreting signals from the sensor 321, generating subject data and transmitting this to a client device or other processing system via the transceiver 325. Accordingly, the electronic processing device is typically a microprocessor, microcontroller, microchip processor, logic gate configuration, firmware optionally associated with implementing logic such as an FPGA (Field Programmable Gate Array), or any other electronic device, system or arrangement.

In use the monitoring device 320 is coupled to or placed in contact with a patch 310, including the substrate 311 and microstructures 313, which are coupled to the sensor 321 and/or signal generator 323 via connections 312. The connections could include physical conductive connections, such as conductive tracks, although this is not essential and alternatively wireless connections could be provided, such as inductive coupling or radio frequency wireless connections. However, this is not essential, and alternatively the monitoring device might be integrated with the patch, so that the monitoring device and patch are a single unitary device.

In this example, the patch further includes anchor microstructures 314 that are configured to penetrate into the dermis and thereby assist in securing the patch to the subject.

In one example, the monitoring device operates as part of a distributed architecture, an example of which will now be described with reference to FIG. 4.

In this example, one or more processing systems 410 are coupled via communications networks 440, and/or one or more local area networks (LANs), to a number of client devices 430 and monitoring devices 420. The monitoring devices 420 could connect directly to the networks, or could be configured to connect to a client device 430, which then provides onward connectivity to the networks 440. It will be appreciated that the configuration of the networks 440 are for the purpose of example only, and in practice the processing systems 410, client devices 430 and monitoring devices 420 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, NFC, or the like.

In one example, each processing system 410 is configured to receive subject data from a monitoring device 420 or client device 430, and analyze the subject data to generate one or more health status indicators, which can then be provided to a client device 430 or monitoring device 420 for display. Whilst the processing system 410 is a shown as a single entity, it will be appreciated that the processing system 410 can be distributed over a number of geographically separate locations, for example by using processing systems 410 and/or databases that are provided as part of a cloud based environment. However, the above described arrangement is not essential and other suitable configurations could be used.

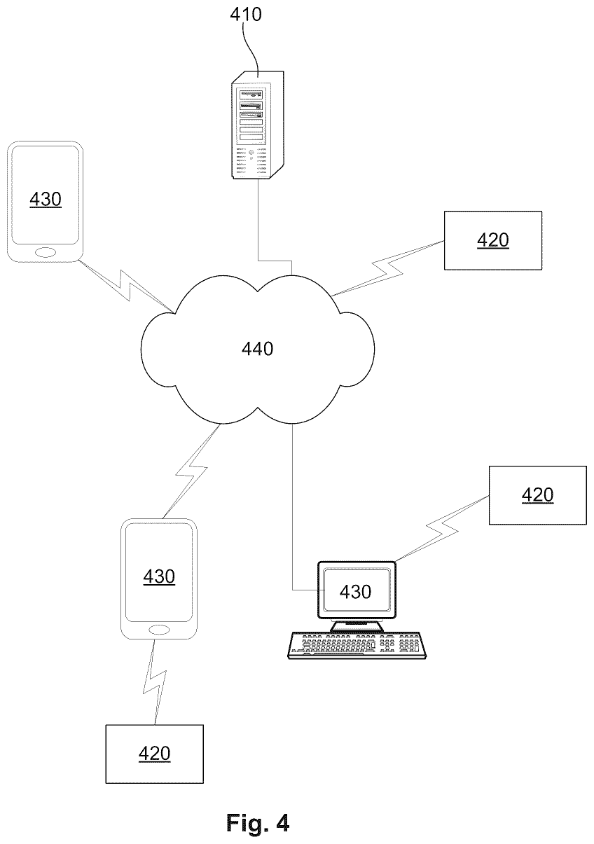

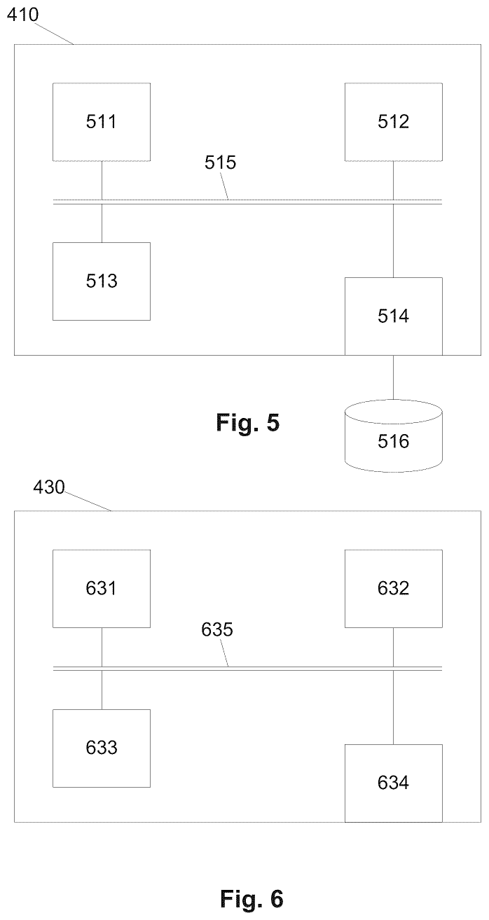

An example of a suitable processing system 410 is shown in FIG. 5.

In this example, the processing system 410 includes at least one microprocessor 511, a memory 512, an optional input/output device 513, such as a keyboard and/or display, and an external interface 514, interconnected via a bus 515 as shown. In this example the external interface 514 can be utilized for connecting the processing system 410 to peripheral devices, such as the communications network 440, databases 516, other storage devices, or the like. Although a single external interface 514 is shown, this is for the purpose of example only, and in practice multiple interfaces using various methods (eg. Ethernet, serial, USB, wireless or the like) may be provided.

In use, the microprocessor 511 executes instructions in the form of applications software stored in the memory 512 to allow the required processes to be performed. The applications software may include one or more software modules, and may be executed in a suitable execution environment, such as an operating system environment, or the like.

Accordingly, it will be appreciated that the processing system 410 may be formed from any suitable processing system, such as a suitably programmed client device, PC, web server, network server, or the like, and in one example, could be part of a distributed system, such as a cloud computing arrangement. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement.

An example of a suitable client device 430 is shown in FIG. 6.

In one example, the client device 430 includes at least one microprocessor 631, a memory 632, an input/output device 633, such as a keyboard and/or display, and an external interface 634, interconnected via a bus 635 as shown. In this example the external interface 634 can be utilized for connecting the client device 430 to peripheral devices, such as the communications networks 440, databases, other storage devices, or the like. Although a single external interface 634 is shown, this is for the purpose of example only, and in practice multiple interfaces using various methods (eg. Ethernet, serial, USB, wireless or the like) may be provided.

In use, the microprocessor 631 executes instructions in the form of applications software stored in the memory 632 to allow communication with the processing system 410 and/or monitoring device 420.

Accordingly, it will be appreciated that the client devices 430 may be formed from any suitable processing system, such as a suitably programmed PC, Internet terminal, lap-top, or hand-held PC, and in one preferred example is either a tablet, or smart phone, or the like. However, it will also be understood that the client devices 430 can be any electronic processing device such as a microprocessor, microchip processor, logic gate configuration, firmware optionally associated with implementing logic such as an FPGA (Field Programmable Gate Array), or any other electronic device, system or arrangement.

It will be appreciated that there is significant similarity between the client device 430 and monitoring device 420, and that in practice the functionality of these devices could be implemented using a single device, such as a smartphone or similar. In this example, the smartphone could be configured to perform measurements using a Bluetooth, NFC or other similar interface, to communicate with electronics on the patch, allowing the measurement to be performed.

Examples of the processes for performing measurements and generating indicators will now be described in further detail. For the purpose of these examples it is assumed that one or more processing systems 410 act to analyze received subject data and generate resulting indicators. Measurements are performed by the monitoring devices 420 with subject data being transferred to the processing systems 410, via the client devices 430, although it will be appreciated that measurements could be performed by the client device 430, or data transferred to the servers 410 directly from the monitoring devices 420. The processing system 410 is therefore typically a server (and will hereinafter be referred to as a server) which communicates with the client device 430 and/or monitoring device 420, via a communications network 440, or the like, depending on the particular network infrastructure available.

To achieve this the server 410 typically executes applications software for hosting webpages, as well as performing other required tasks including storing, searching and processing of data, with actions performed by the server 410 being performed by the processor 511 in accordance with instructions stored as applications software in the memory 512 and/or input commands received from a user via the I/O device 513, or commands received from a client device 430 and/or monitoring device 420.

It will also be assumed that the user interacts with the server 410 via a GUI (Graphical User Interface), or the like presented on a client device 430 and/or monitoring device 420. Actions performed by the client device 430 are performed by the processor 631 in accordance with instructions stored as applications software in the memory 632 and/or input commands received from a user via the I/O device 602. Similarly actions performed by the monitoring device 420 are performed by the processor 332 in accordance with instructions stored as applications software in the memory 324 and/or input commands received from a user via the I/O device 325.

However, it will be appreciated that the above described configuration assumed for the purpose of the following examples is not essential, and numerous other configurations may be used. It will also be appreciated that the partitioning of functionality between the monitoring devices 420, client devices 430, and the server 410 may vary, depending on the particular implementation.

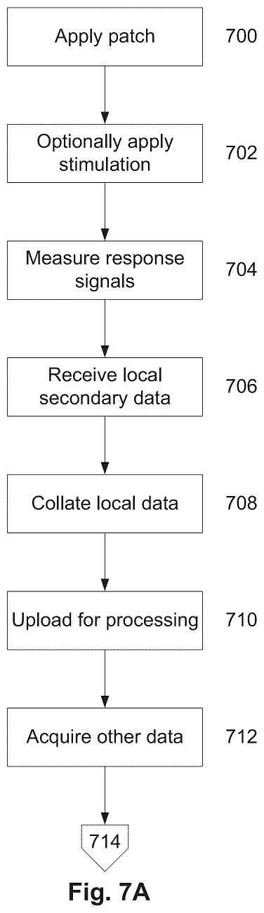

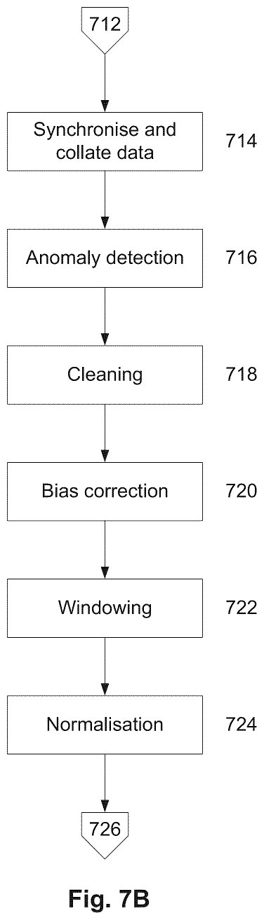

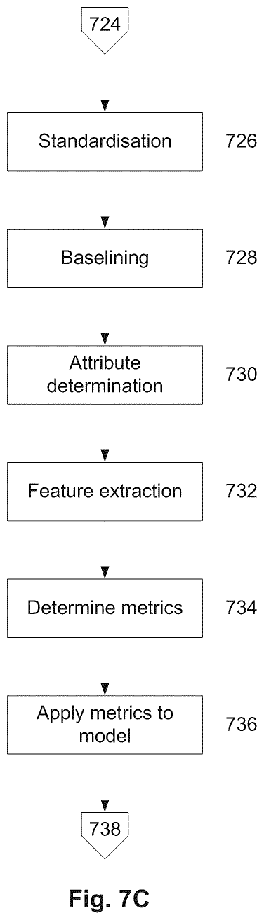

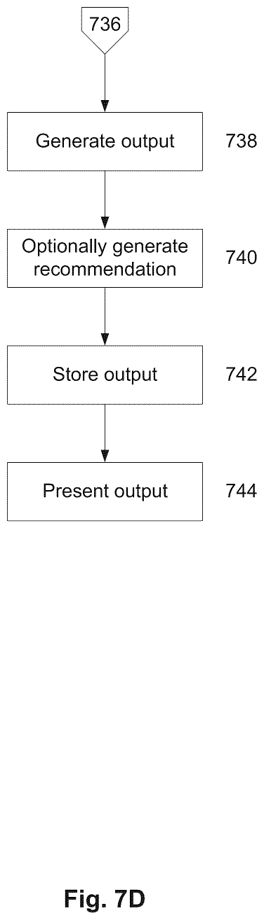

An example of a process for performing measurements on a subject will now be described in more detail with reference to FIGS. 7A to 7D.

In this example, the patch is applied to the subject at step 700 so that the microstructures on the patch penetrate the epidermis. In one example, the microstructures are electrically conductive allowing the epidermis to be electrically interrogated to measure the electrical properties of the epidermis between microstructures. However, in another example, different patches can be applied to different parts of the body, allowing measurements to be performed between devices located on different parts of the body. Examples of electrical properties that can be measured include impedance, conductivity, capacitance, resistance, scattering parameters, ion selectively measurements, or the like. It will also be appreciated that electrical signals from the body itself, such as ECG, EMG or EEG signals, could also be measured, whilst measurements could also be made of the electrical properties of sweat or other bodily fluids.

Alternatively, the device might have microstructures that are functionalized with chemicals that can bind to specific biomarker molecules present in the epidermis and result in electrical measurements correlating with the presence of those biomarkers. The chemicals used to bind to target analytes might be antibodies, aptamers or polymers.

Another measurement regime might involve the use of photoplethysmography to determine blood flow and/or blood oxygenation. Specific algorithms can also use this kind of data to extract information like pulse rate, arterial elasticity, respiratory rate or heart rate variability. Another measurement regime might involve the use of optical measurements such as NIR LEDs to interrogate water content or other molecular content of the tissue. Finally, any combination of the above measurement regimes might be used.

In any event, it will be appreciated that the monitoring device 420 can be used to trigger the application of stimulation at step 702, assuming this is required by the particular sensing being performed, with response signals being measured at step 704.

Additional local secondary data might also be acquired by the monitoring device 420 at step 706. Such local secondary data might include measurements made or received by the monitoring device from secondary sensors, and might include measurements such as motion (through accelerometry), ambient temperature, skin temperature, skin surface impedance, humidity, location (GPS) etc. Additionally, inputs from a user such as fluid intake, weight changes, calorie intake could be recorded in an application or web site and form other streams of data that are useful in processing data from the sensor into the desired outputs. User parameters may also be entered including weight, height, age, sex, ethnicity etc.

The monitoring device 420 can include memory or temporary storage to store measurements allowing these to be collated at step 708 until they can be sent to another device for processing. Alternatively, any/all of the subsequent steps could be processed on the device itself. The device may also have inbuilt capabilities to communicate information to a user such as lights, speakers, LED or LCD displays, or haptic feedback capabilities.

It is also possible that the monitoring device 420 might be a system of multiple wearable devices each with a unique and complimentary set of the measurement regimes mentioned above.

In addition to data from secondary sensors, secondary data may be acquired from other sources, including other publicly available data or data measured by other devices, such as weather data or data measured from other medical devices or wearables such as pulse oximeters etc.

Once the sensor data has been collected this can be collated at step 708 to form part of the subject data, which in its simplest form just includes the sensor data and any secondary sensor data, but may also include additional information, such as a subject or patch identifier, or similar. This portion of the subject data is then typically uploaded for further processing at step 710, with this step typically involving the portion of the subject data being encrypted and/or uploaded over an encrypted and authenticated channel.

The portion of the subject data may be sent via any method of data transfer to the client device 430, which might be a smartphone, tablet, laptop, personal computer or any other device with onboard storage and processing, or to the server 410. In either case, the client device 430 may also be involved in any/all subsequent data processing steps and could also host an application that displays sensor outputs to a user, recommends actions to a user and collects useful information about a user (e.g. age, gender, height, fluid intake etc.). The client device 430 may also be the device running applications that can accept user inputs containing information about the user's behaviors that are used as additional streams of data for the model or model evaluation.

Alternatively, and more typically, the data may be uploaded to the client device 430, tagged with identifiers or similar, and then uploaded to the server 410 for storage and analysis.

Any data that is collected may need to be sent through networks to a storage/processing device/server and this sensitive data would need to be encrypted. The data could be encrypted by firmware and/or software on a monitoring device or by the client device, or any combination of these.

Assuming the portion of the subject data has now been received by the server 410, at step 712, other data is acquired as needed. The other data can include other secondary data, such as subject trait data, or learned subject parameters. Such secondary data can include historical data, which is typically accessed by the server, allowing historical data to be retrieved for use in later data processing. For example, data can be retrieved from Electronic Medical Records Data, which may be packaged with reference historical electronic medical records (EMR) which would be used in subsequent data processing. Medical records might contain information about user pathology or previously made physiological measurements in a standardized form that can be used in the model or model evaluation. Additionally and/or alternatively, secondary data could be acquired from other sources, for example, through manual input of data by a user.

In addition to secondary data, the other data can include context data, which is typically data relevant to the context in which the measurements have been performed. The context data could include local environment data, such as air temperature, pressure or humidity readings captured by one or more sensors, or the like. The context data might also include external context data such as weather data, details of a subject work schedule, or similar, and this information might be retrieved from external sources, such as a bureau of meteorology, a calendar, or similar.

Once the received subject data, the secondary data, and context data have been collected these form the full suite of subject data, which can then be analyzed.

As part of this process, the subject data may need to be chronologically synchronized at step 714, making sure that events in the different data temporally align, for example ensuring an event in the sensor data aligns with a corresponding event in the secondary sensor data. The synchronization is typically achieved by means of an internal clock within the monitoring device 420 that can be used to tag all measurements with a time-stamp. However, it will be appreciated that other approaches could be used, such as using a particular stimulatory signal measurable by different sensors, to thereby temporally align the data, or by identifying common events measured by different sensors, such as by aligning movement with spikes in heart activity or similar. These time stamped data streams would be typically be synchronized by the monitoring device, although this is not essential and synchronization can be performed by the server 410.

At this point, the some or all of the subject data might be stored, for example, adding the sensor data to historical data, allowing this to be subsequently accessed and used as required, for example in performing longitudinal monitoring. Thus, in a clinical setting, it is possible that measurements/outputs made by the sensor device could become EMR themselves that subsequent sensor measurements could reference.

Thus, it will be appreciated that the subject data and electronic medical records might be sent to a central location or distributed cloud service for storage and processing. This facility could be a server, computational or storage cloud service, computer, smartphone or any computational device with sufficient capabilities. This is also where the output of data processing would be stored.

At step 716, subject data can be checked by the server 410 for anomalies using anomaly detection algorithms. Anomalies might be sudden changes in magnitudes that could not possibly be physiological in nature, outliers or spikes in magnitudes, missing values etc. Anomalies might be flagged by special algorithms such as DBSCAN. Anomalies might also be identified in the context of other collected data. For example, extreme values that correlate with a lot of motion measured by accelerometers. Meta anomaly detection algorithms may also be used to check for patterns in anomalies that might indicate problems with hardware and flag to the user that the sensor is not working properly.

Any data determined to be anomalous according to customized algorithms can be flagged for cleaning at step 718. In this step, cleaning algorithms appropriately process the anomalies flagged in the previous step along with missing or corrupted data. This might involve deleting missing/extreme values or interpolating to replace those values. Alternatively, entire portions of sensor data might be removed or truncated to exclude an anomaly or missing value/s. However, it will be appreciated that anomalies might also be indicative of the subject's physiological state and hence not all anomalies will be cleaned, and instead these might be flagged for generation of metrics or the like.

At step 720, bias correction and/or calibration can be performed so that systematic errors or known biases in the measurements made by the sensor would be corrected. These may correct for individual sensor characteristics, and might be dependent on other variables. For example, one measurement might have a known bias linked to ambient temperature and so if secondary sensor data includes an indication of the ambient temperature, this allows the sensor data to be corrected. Some of these corrections may be in the form of filtering techniques and/or could be done based on motion/fluid shifts or known baseline drifts.

It will be noted that because the sensor is small and typically inferring physiological states from a much smaller area, it should be resilient to more macroscopic physiological changes (such as edema). This means that the data generated by sensor may have less bias to be corrected than other similar sensors.

In order to facilitate the processing of continuous measurements at step 722, a windowing process can optionally be performed in order to segment the data into discrete windows or overlapping windows. Each window would typically be a limited number of samples used for making a prediction of a physiological state. The windows could be defined chronologically (for example segmenting the data into 5-minute windows), by samples (each window is x number of measurements), by events (a significant change in the baseline measurements might define a window boundary) or by a combination of these. Outputs from the windowing can then be used in generating attributes and features used as inputs for the computational model. However, it should also be noted that windowing may not be required, and instead techniques might be used that can assimilate a continuous data stream and make continuous predictions without windowing, for example using exponentially weighted moving averages (EWMA), Bayesian filtering, or the like.

At step 724, normalization can be performed based on the magnitudes recorded in each window. This can assist in extracting features such as relative changes in magnitude rather than absolute magnitudes. Additionally, other data in the pipeline might inform the process of normalization, for example the range of historical data for a single subject or multiple subjects.

At step 726 standardization can be performed according to information collected about the subject, such as physiological attributes, including the subject's height, age, gender, or the like. This can additionally and/or alternatively take into account historical data to establish a baseline measurement for that user, which is then used in a baselining process at step 728. In one example, this involves using the subject's historical data to inform the model of what the average or typical value/s is/are for that subject in one or multiple physiological states. Baselining might occur by adjusting the raw values of the data, adjusting values of the data after any point in the processing, or by attaching a flag to the data containing useful information for displaying, adjusting or interpreting the model output. Baselining may additionally and/or alternatively be done through a machine learning model that is refined through research and training data generated from multiple subjects.

The preceding steps 716 to 728 might each be preceded by signal processing to transform the data and/or extract useful attributes from the data. These signal processing techniques might involve blind source separation algorithms such as independent component analysis or principal component analysis. The techniques might also involve analysis of rates of change in curves, or the integration or summation of quantities over an interval of time. It will also be appreciated that some or all of these data processing steps may not be required, and/or could be performed in different orders, depending on the nature of the data being analyzed and/or the preferred implementation. For example, heart rate measurements may require different processing is performed compared to impedance measurements, whilst heart rate information collected via a heart rate sensor may require different processing compared to ECG measurements. The above described processing steps should therefore be considered as illustrative and not limiting.

Once all the data has been processed as required, important attributes from the data can optionally be collected at step 730. These attributes form a concise package of information important to the model and might include statistical summaries like mean, variance, skew, kurtosis, or the like, as well as numerical values generated through signal processing or applying equivalent circuit fitting to the data. In this case, at step 732 the attributes can be processed or combined using feature engineering algorithms to generate features. It will be appreciated that some of the attributes may not need to be changed while others may undergo some sort of processing or arithmetical combinations with other features.

The generated attributes and features, together with the raw or pre-processed data, form a pool of metrics, allowing relevant metrics to be selected at step 734, allowing these to be applied as model inputs at step 736, thereby generating a model output at step 738.

However, it will be appreciated that explicitly generating attributes and features is not essential, and that additionally and/or alternatively models may be used to analyze raw or pre-processed data without necessarily requiring that attributes and/or features are first identified.

In any event, assuming a model is used to analyze metrics, the metric inputs are analyzed using a model to predict a physiological status. The algorithms used in this step typically comprise any number of machine learning or statistical inference algorithms including but not limited to: polynomial regression, logistic regression, clustering algorithms, neural networks, random forest, decision trees, Bayesian algorithms, Gaussian processes or ensemble methods. The model embodies relationships between combinations of different metric values and corresponding physiological states, so that when metrics are analyzed using the model, the model output is indicative of a particular physiological state.

The simplest form of model output would involve generating a classification. In this approach, a user is classified into a limited number of arbitrarily defined categories. For example, a user might be classified as overhydrated, euhydrated, dehydrated or severely dehydrated or the like. The classification could be presented in various formats including but not limited to a traffic light system or any other format conducive to easy understanding.

In another example, a model with better performance might be able to express measurements of physiology as an arbitrary index. For example, on an arbitrary scale of 1-10 for hydration status.

Alternatively, a very sophisticated model could be used to make sufficiently accurate estimates of an underlying physiological value that it could be said to measure this. For example, blood osmolarity or total body water could be estimated, or in the case of a biomarker sensor an example might be estimating blood levels of a biomarker, such as troponin.

The model output might be used to generate a recommendation, so for example if the model output indicates the subject is underhydrated, the recommendation might be for the user to increase their hydration levels. Such recommendations would typically be generated based on rules, or similar, which can set out different recommendations that should be associated with different model outputs. The recommendations could also be more sophisticated and could take into account a subject history, details of the subject physiology, activities being undertaken or similar.

In this regard, it will be understood that the indicator, whilst typically reflecting a current status of the subject, could also be predictive, for example, identifying when it is predicted that a certain physiological state might occur, based on current sensor readings and context. For example, with an athlete participating in a sporting activity, this could be used to predict when the athlete might become dehydrated, allowing the athlete to take preventative action in advance.

Once the output has been generated, this will typically be stored at step 742. In general, the output would be stored together with other data generated by process, such as the sensor data, subject data and any intermediary forms of data generated during the processing. In one example, the data can be stored as part of a subject profile, medical history, medical records, or the like.

At step 744, the output and/or any recommendation can be presented for viewing. This could be achieved, for example, by communicating the output and any associated recommendation to a client device 430 and/or the monitoring device 420, allowing this to be viewed by the subject. Display to user might involve showing them the direct output of the model or maybe simplifying the output into something easier to understand. Graphs of model output as a function of time might be generated to aid the user's understanding and experience. Based on the information users have displayed to them they may act, which could possibly be recorded into applications to inform/reevaluate the model. User actions could also impact subsequent measurements made by the sensor device forming a biofeedback-style loop. Some of such inputs could be water, calorie intake, medicine, activity etc.

Additionally and/or alternatively, this could be displayed to an overseeing individual, such as a medical practitioner, allowing this to be actioned as needed. Thus, it will be appreciated that outputs of the model and any other relevant information could be displayed to clinicians for the purpose of medical decision making, such as making diagnostic decisions to identify and characterize pathological states.

Historical data generated by the sensor could be packaged for use in research and could be sold/provided to commercial/academic organizations. Research into the data may allow it to be associated with specific pathologies thus broadening the clinical/wellness applications of the device. In this instance, statistical validation might be performed to ensure the historical data is meets requirements for accuracy as needed, depending on the intended application.

In another example, the output might be used to trigger an intervention, as an input to a control system that is configured to administer medication, or the like. Thus, clinical data could be used to drive therapeutics by, for example, informing dosing of pharmacological interventions or dynamically informing medical devices such as smart IV or dialysis machines, or the like.

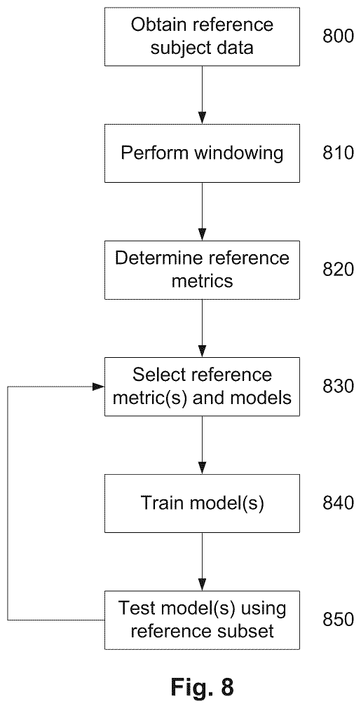

The above described approach uses one or more models in order to determine a physiological state indicator, and an example of a process for generating and/or fitting such a model will now be described with reference to FIG. 8.

In this example, reference subject data is obtained at step 800, which is based on subject data collected from reference subjects using the above described process. At step 810 the reference subject data is analyzed to perform windowing, with reference metrics being generated at step 820.

Steps 800 to 820 are largely analogous to steps 700 to 734 described above, and it will therefore be appreciated that these can be performed in a largely similar manner, and hence will not be described in further detail.

In contrast to collection and analysis of subject data however, as the reference subject data is used in training a model, the reference subject data is also indicative of one or more physiological states.

Additionally, when using the reference subject data to train the model, it will be typical to determine reference metrics for all available metrics, including all possible attributes and features, rather than just selected ones of the metrics, allowing this to be used in order to ascertain which of the metrics are most useful in assessing physiological states. Nevertheless, the reference metrics used are as outlined above.

At step 830 a combination of the reference metrics and one or more generic models are selected, with the reference metrics and identified physiological states for a plurality of reference subjects being used to train the model(s) at step 840.

The nature of the model and the training performed can be of any appropriate form. The model could comprise any one or more of biophysical models, computational models, statistical models and biochemical models. The algorithms used in training could include any one or more of decision tree learning, random forest, logistic regression, association rule learning, artificial neural networks, deep learning, inductive logic programming, support vector machines, clustering, Bayesian networks, Bayesian computation, Bayesian data assimilation, reinforcement learning, representation learning, similarity and metric learning, genetic algorithms, rule-based machine learning, fitting of statistical models, fitting of dynamic causal models, learning classifier systems, or the like. As such schemes are known, these will not be described in any further detail. In one example, this can include training a single model to determine the physiological state based on a combination of metrics, although this is not essential, and alternatively this will involve determining a multiple models for different physiological states.

Accordingly, the above described process provides a mechanism to develop and train a model that can be used in generating a physiological state indicator using the process described above.

In addition to simply generating the model, the process typically includes testing the model at step 850 to assess the quantitative accuracy or discriminatory performance of the trained model. Such testing is typically performed using a subset of the reference subject data, and in particular, different reference subject data to that used to train the model, to avoid bias. The testing is used to ensure the computational model provides sufficient quantitative accuracy or discriminatory performance. In this regard, discriminatory performance is typically based on an accuracy, sensitivity, specificity and AUROC, with a discriminatory performance of at least 70% being required in order for the model to be used.

It will be appreciated that if the model meets the requirements for discriminatory performance, it can then be used in determining a physiological indicator using the process outlined above with respect to FIG. 1. Otherwise, the process returns to step 830 allowing different metrics and/or models to be selected, or to allow additional data to be collected to perform training, with further training and testing then being repeated as required.

The above described process has focused on the generation of computational models for performing classification, but it will be appreciated that other appropriate approaches could be used for deriving and training or fitting other forms of model.

Furthermore, it will be appreciated that as additional subject data is collected, this can be used to refine the models over time, so that the models become progressively more accurate. This may include refinement or fitting of models for individual users, and/or refinement of shared models for a population of users.

Thus, in one example, the one or more processing devices select a plurality of reference metrics, typically selected as a subset of each of the metrics listed above, train one or more computational model(s) using the plurality of reference metrics, test the computational model(s) to determine a discriminatory performance of the model and if the discriminatory performance of the model falls below a threshold then selectively retrain the computational model(s) using a different plurality of reference metrics and/or train a different computational model(s). Accordingly, it will be appreciated that the above described process can be performed iteratively utilizing different metrics and/or different computational models until a required degree of discriminatory power is obtained.

As an alternative, the one or more processing devices can select a plurality of combinations of reference metrics, train a plurality of models using each of the combinations, test each computational model to determine a performance of the model and select the model with the highest performance for use in determining a physiological state of interest.

In addition to use the metrics to train the models, the training can also be performed taking into account reference subject attributes, so that models are specific to respective reference subject attributes or can take the subject attributes into account when determining the physiological state of interest. In one example, this process involves having one or more processing devices perform clustering using the using the reference subject attributes to determine clusters of reference subjects having similar reference subject attributes, for example using a clustering technique such as k-means clustering, and then training the computational model at least in part using the reference subject clusters. For example clusters of reference individuals suffering from a particular physiological condition could be identified, with this being used to train a computational model to identify the physiological condition. It will be appreciated however that any suitable technique could be used.

In a further example, the processing devices develop the model by performing one or more of feature analysis and down-selection, correlation and univariate statistical separability tests and dimensionality reduction. Thus, for example, this allows for the calculation of multiple metrics, and multiple models, with those refined, evolved or eliminated depending on their discriminatory power. Such refining can be performed using one or more of cross-validation performance, hyperparameter validation, learning curve analysis or metric relevance across models.

Accordingly, the above described techniques provide a mechanism for training one or more computational models to discriminate between different physiological states using a variety of different metrics, and then using the model(s) to generates physiological state indicators indicative of the likelihood of a subject having a particular physiological state.

In one example, where there is subject data that could be used to evaluate the model's accuracy, the model could further be evaluated and updated using that data. For example, when the device is used in clinical trials or in clinical settings, or when sufficiently accurate user inputs (such as fluid intake) are available. In these cases, the model's output could be compared with actual hydration measurements such as plasma osmolarity or an estimate of hydration based on fluid intake.

As mentioned above, at least some of microstructures include an electrode, which can be used to apply electrical signals to a subject, measure intrinsic or extrinsic response electrical signals, for example measuring ECG or impedances. The microstructures could be made from a metal or other conductive material, so that the entire microstructure constitutes the electrode, or alternatively the electrode could be coated or deposited onto the microstructure, for example by depositing a layer of gold to form the electrode. The electrode material could include any one or more of gold, silver, colloidal silver, colloidal gold, colloidal carbon, carbon nano materials, platinum, titanium, stainless steel, or other metals, or any other biocompatible conductive material.

In a further example, the microstructure could include an electrically conductive core or layer covered by a non-conductive layer (insulating), with openings providing access to the core to allow conduction of electrical signals through the openings, to thereby define electrodes. In one example, the insulating layer extends over part of a surface of the microstructure, including a proximal end of the microstructure adjacent the substrate. The insulating layer could extend over at least half of a length of the microstructure and/or about 90 μm of a proximal end of the microstructure, and optionally, at least part of a tip portion of the microstructure. In one specific example, this is performed so the non-insulating portion is located in the epidermis, so stimulatory signals are applied to and/or response signals received from, the epidermis.

The insulating layer could also extend over some or all of a surface of the substrate. In this regard, in some examples connections are formed on a surface of the substrate, in which case a coating, and in particular a dielectric coating such as Parylene, could be used to isolate these from the subject. For example, electrical tracks on a surface of the substrate could be used to provide electrical connections to the electrodes, with an insulating layer being provided on top of the connections to ensure the connections do not make electrical contact with the skin of the subject, which could in turn adversely affect measured response signals. For example, this prevents electrical contact with the skin surface, in turn preventing surface moisture, such as sweat, from influencing the measurements.

The microstructures could have a range of different shapes and could include ridges, needles, plates, blades, or similar. In this regard, the terms plates and blades are used interchangeably to refer to microstructures having a width that is of a similar order of magnitude in size to the length, but which are significantly thinner. The microstructures can be tapered to facilitate insertion into the subject, and can have different cross-sectional shapes, for example depending on the intended use. The microstructures typically have a rectangular cross sectional shape and may include shape changes along a length of the microstructure. For example, microstructures could include a shoulder that is configured to abut against the stratum corneum to control a depth of penetration and/or a shaft extending to the tip, with the shaft being configured to control a position of the tip in the subject and/or provide a surface for an electrode. In another example, the microstructures have bifurcated or angled tips.

Other example shapes include circular, rectangular, cruciform shapes, square, rounded square, rounded rectangular, ellipsoidal, or the like, which can allow for increased surface area, which is useful when coating microstructures to maximize the coating volume and hence the amount of payload delivered per microstructure, although it will be appreciated that a range of other shapes could be used. Microstructures can have a rough or smooth surface, or may include surface features, such as pores, raised portions, serrations, or the like, which can increase surface area and/or assist in penetrating or engaging tissue, to thereby anchor the microstructures within the subject. This can also assist in reducing biofouling, for example by prohibiting the adherence and hence build-up of biofilms. The microstructures might also be hollow or porous and can include an internal structure, such as holes or similar, in which case the cross sectional shape could also be at least partially hollow. In particular embodiments, the microstructures are porous, which may increase the effective surface area of the microstructure. The pores may be of any suitable size to allow an analyte of interest to enter the pores, but exclude one or more other analytes or substances, and thus, will depend on the size of the analyte of interest. In some embodiments, the pores may be less than about 10 μm in diameter, preferably less than about 1 μm in diameter.

In one example, the microstructures have a rounded rectangular shape when viewed in cross section through a plane extending laterally through the microstructures and parallel to but offset from the substrate. The microstructures may include shape changes along a length of the microstructure. For example, microstructures could include a shoulder that is configured to abut against the stratum corneum to control a depth of penetration and/or a shaft extending to the tip, with the shaft being configured to control a position of the tip in the subject and/or provide a surface for an electrode.

Different microstructures could be provided on a common substrate, for example providing different shapes of microstructure to achieve different functions. In one example, this could include performing different types of measurement. In other examples, microstructures could be provided on different substrates, for example, allowing sensing to be performed via microstructures on one patch and delivery of therapy to be performed via microstructures on a different patch. In this example, this could allow a therapy patch to be replaced once exhausted, whilst a sensing patch could remain in situ. Additionally, measurements could be performed between patches, for example, performing whole of body impedance measurements between patches provided at different locations on a subject.

Additionally and/or alternatively anchor microstructures could be provided, which can be used to anchor the substrate to the subject. In this regard, anchor microstructures would typically have a greater length than that of the microstructures, which can help retain the substrate in position on the subject and ensure that the substrate does not move during the measurements or is not being inadvertently removed. Anchor microstructures can include anchoring structures, such as raised portions, which can assist with engaging the tissue, and these could be formed by a shape of the microstructure and/or a shape of a coating. Additionally, the coating could include a hydrogel or other similar material, which expands upon expose to moisture within the subject, thereby further facilitating engagement with the subject. Similarly the microstructure could undergo a shape change, such as swelling either in response to exposure to substances, such as water or moisture within the subject, or in response to an applied stimulation. When applied to skin, the anchor microstructures can enter the dermis, and hence are longer than other microstructures, to help retain the substrate in place, although it will be appreciated that this is not essential and will depend upon the preferred implementation. In other examples the anchor microstructures are rougher than other microstructures, have a higher surface friction than other microstructures, are blunter than other microstructures or are fatter than other microstructures.

In a further example, at least part of the substrate could be coated with an adhesive coating in order to allow the substrate and hence patch, to adhere to the subject.

As previously mentioned, when applied to skin, the microstructures typically enter the viable epidermis and in one example, do not enter the dermis, although in other examples, may enter the dermis. But this is not essential, and for some applications, it may be necessary for the microstructures to enter the dermis, for example projecting shortly through the viable epidermis/dermis boundary or entering into the dermis a significant distance, largely depending on the nature of the sensing being performed. In one example, for skin, the microstructures have a length that is at least one of less than 2500 μm, less than 1000 μm, less than 750 μm, less than 600 μm, less than 500 μm, less than 400 μm, less than 300 μm, less than 250 μm, greater than 100 μm, greater than 50 μm and greater than 10 μm, but it will be appreciated that other lengths could be used. More generally, when applied to a functional barrier, the microstructures typically have a length greater than the thickness of the functional barrier, at least 10% greater than the thickness of the functional barrier, at least 20% greater than the thickness of the functional barrier, at least 50% greater than the thickness of the functional barrier, at least 75% greater than the thickness of the functional barrier and at least 100% greater than the thickness of the functional barrier.

In another example, the microstructures have a length that is no more than 2000% greater than the thickness of the functional barrier, no more than 1000% greater than the thickness of the functional barrier, no more than 500% greater than the thickness of the functional barrier, no more than 100% greater than the thickness of the functional barrier, no more than 75% greater than the thickness of the functional barrier or no more than 50% greater than the thickness of the functional barrier. This can avoid deep penetration of underlying layers within the body, which can in turn be undesirable, and it will be appreciated that the length of the microstructures used will vary depending on the intended use, and in particular the nature of the barrier to be breached, and/or signals to be applied or measured. The length of the microstructures can also be uneven, for example, allowing a blade to be taller at one end than another, which can facilitate penetration of the subject or functional barrier.

Similarly, the microstructures can have different widths depending on the preferred implementation. Typically, the widths are at least one of less than 25% of the length, less than 20% of the length, less than 15% of the length, less than 10% of the length, or less than 5% of the length. Thus, for example, when applied to the skin, the microstructures could have a width of less than 50 μm, less than 40 μm, less than 30 μm, less than 20 μm or less than 10 μm. However, alternatively, the microstructures could include blades, and could be wider than the length of the microstructures. In some example, the microstructures could have a width of less than 50000 μm, less than 40000 μm, less than 30000 μm, less than 20000 μm, less than 10000 μm, less than 5000 μm, less than 2500 μm, less than 1000 μm, less than 500 μm or less than 100 μm. In blade examples, it is also feasible to use microstructures having a width substantially up to the width of the substrate.

In general the thickness of the microstructures is significantly lower in order to facilitate penetration and is typically less than 1000 μm, less than 500 μm, less than 200 μm, less than 100 μm, less than 50 μm, less than 20 μm, less than 10 μm, at least 1 μm, at least 0.5 μm or at least 0.1 μm. In general the thickness of the microstructure is governed by mechanical requirements, and in particular the need to ensure the microstructure does not break, fracture or deform upon penetration. However, this issue can be mitigated through the use of a coating that adds additional mechanical strength to the microstructures.

In one specific example, for epidermal sensing, the microstructures have a length that is less than 300 μm, greater than 50 μm, greater than 100 μm and about 150 μm, and, a width that is greater than or about equal to a length of the microstructure, and is typically less than 300 μm, greater than 50 μm and about 150 μm. In another example, for dermal sensing, the microstructures have a length that is less than 450 μm, greater than 100 μm, and about 250 μm, and, a width that is greater than or about equal to a length of the microstructure, and at least of a similar order of magnitude to the length, and is typically less than 450 μm, greater than 100 μm, and about 250 μm. In other examples, longer microstructures could be used, so for example for hyperdermal sensing, the microstructures would be of a greater length. The microstructures typically have a thickness that is less than the width, significantly less than the width and of an order of magnitude smaller than the width. In one example, the thickness is less than 50 μm, greater than 10 μm, and about 25 μm, whilst the microstructure typically includes a flared base for additional strength, and hence includes a base thickness proximate the substrate that is about three times the thickness, and typically is less than 150 μm, greater than 30 μm and about 75 μm. The microstructures typically have a tip has a length that is less than 50% of a length of the microstructure, at least 10% of a length of the microstructure and more typically about 30% of a length of the microstructure. The tip further has a sharpness that is at least 0.1 μm, less than 5 μm and typically about 1 μm.

In one example, the microstructures have a relatively low density, such as less than 10000 per cm², such as less than 1000 per cm², less than 500 per cm², less than 100 per cm², less than 10 per cm² or even less than 5 per cm². The use of a relatively low density facilitates penetration of the microstructures through the stratum corneum and in particular avoids the issues associated with penetration of the skin by high density arrays, which in turn can lead to the need for high powered actuators in order for the arrays to be correctly applied. However, this is not essential, and higher density microstructure arrangements could be used, including less than 50,000 microstructures per cm², less than 30,000 microstructures per cm², or the like. As a result, the microstructures typically have a spacing that is less than 20 mm, less than 10 mm, less than 1 mm, less than 0.1 mm or less than 10 μm. It should be noted that in some circumstances, microstructures are arranged in pairs, with the microstructures in each pair having a small spacing, such as less than 10 μm, whilst the pairs have a great spacing, such as more than 1 mm, in order to ensure a low overall density is maintained. However, it will be appreciated that this is not essential, and higher densities could be used in some circumstances.

In one specific example, the microstructures have a density that is less than 5000 per cm², greater than 100 per cm², and about 600 per cm², leading to a spacing of less than 1 mm, more than 10 μm, and about 0.5 mm, 0.2 mm or 0.1 mm.

In one example, the microstructures include plates having a substantially planar face having an electrode thereon. The use of a plate shape maximizes the surface area of the electrode, whilst minimizing the cross sectional area of the microstructure, to thereby assist with penetration of the microstructure into the subject. This also allows the electrode to act as a capacitive plate, allowing capacitive sensing to be performed. In one example, the electrodes have a surface area of at least at least 10 mm², at least 1 mm², at least 100,000 μm², 10,000 μm², at least 7,500 μm², at least 5,000 μm², at least 2,000 μm², at least 1,000 μm², at least 500 μm², at least 100 μm², or at least 10 μm². In one example, the electrodes have a width or height that is up to 2500 μm, at least 500 μm, at least 200 μm, at least 100 μm, at least 75 μm, at least 50 μm, at least 20 μm, at least 10 μm or at least 1 μm. In the case of electrodes provided on blades, the electrode width could be less than 50000 μm, less than 40000 μm, less than 30000 μm, less than 20000 μm, less than 10000 μm, or less than 1000 μm, as well as including widths outlined previously. In this regard, it will be noted that these dimensions apply to individual electrodes, and in some examples each microstructure might include multiple electrodes.

In one specific example, the electrodes have a surface area of less than 200,000 μm², at least 2,000 μm² and about 22,500 μm², with the electrodes extending over a length of a distal portion of the microstructure, optionally spaced from the tip, and optionally positioned proximate a distal end of the microstructure, again proximate the tip of the microstructure. The electrode can extend over at least 25% and less than 50% of a length of the microstructure, so that the electrode typically extends over about 60 μm 90 μm or 150 μm of the microstructure and hence is positioned in a viable epidermis and/or dermis of the subject in use.

In one example, at least some of the microstructures are arranged in groups, such as pairs, with response signals or stimulation being measured from or applied to the microstructures within the group. The microstructures within the group can have a specific configuration to allow particular measurements to be performed. For example, when arranged in pairs, a separation distance can be used to influence the nature of measurements performed. For example, when performing bioimpedance measurements, if the separation between the microstructures is greater than a few millimeters, this will tend the measure properties of interstitial fluid located between the electrodes, whereas if the distance between the microstructures is reduced, measurements will be more influenced by surface properties, such as the presence of materials bound to the surface of the microstructures. Measurements are also influenced by the nature of the applied stimulation, so that for example, current at low frequencies will tend to flow though extra-cellular fluids, whereas current at higher frequencies is more influenced by intra-cellular fluids.

In one particular example, plate microstructures are provided in pairs, with each pair including spaced apart plate microstructures defining substantially planar electrodes in opposition. This can be used to generate a highly uniform field in the subject in a region between the electrodes, and/or to perform capacitive or conductivity sensing of substances between the electrodes. This effect can be further enhanced by providing rows of microstructures, with microstructures within a row being electrically connected so that measurements can be performed between rows of microstructures, thereby increasing the effective electrode surface area. However, this is not essential, and other configurations, such as circumferentially spacing a plurality of electrodes around a central electrode, can be used. Typically the spacing between the electrodes in each group is typically less than 50 mm: less than 20 mm, less than 10 mm, less than 1 mm, less than 0.1 mm or less than 10 μm, although it will be appreciated that greater spacings could be used, including spacing up to dimensions of the substrate and/or greater, if microstructures are distributed across multiple substrates.

Thus, in one specific example, at least some of the microstructures are arranged in pairs or pairs of rows, with response signals being measured between microstructures in the pair or row pair and/or stimulation being applied between microstructures in the pair or row pair. Each pair of microstructures typically includes spaced apart plate microstructures having substantially planar electrodes in opposition and/or spaced apart substantially parallel plate microstructures.

In one example, at least some pairs of microstructures are angularly offset, and in one particular example, are orthogonally arranged. Thus, in the case of plate microstructures, at least some pairs of microstructures extend in different and optionally orthogonal directions. This distributes stresses associated with insertion of the patch in different directions, and also acts to reduce sideways slippage of the patch by ensuring plates at least partially face a direction of any lateral force. Reducing slippage either during or post insertion helps reduce discomfort, erythema, or the like, and can assist in making the patch comfortable to wear for prolonged periods. Additionally, this can also help to account for any electrical anisotropy within the tissue, for example as a result of fibrin structures within the skin, cellular anisotropy, or the like.

In one specific example, adjacent pairs of microstructures are angularly offset, and/or orthogonally arranged, and additionally and/or alternatively, pairs of microstructures can be arranged in rows, with the pairs of microstructures in one row are orthogonally arranged or angularly offset relative to pairs of microstructures in other rows.

In one specific example, when pairs of microstructures are used, a spacing between the microstructures in each pair is typically less than 0.25 mm, more than 10 μm and about 0.1 mm, whilst a spacing between groups of microstructures is typically less than 1 mm, more than 0.2 mm and about 0.5 mm. Such an arrangement helps ensure electrical signals are primarily applied and measured within a pair and reduces cross talk between pairs, allowing independent measurements to be recorded for each pair of microstructures/electrodes.

The microstructures can be configured in order to interact with, and in particular, bind with one or more analytes of interest, allowing these to be detected. Specifically, in one example, binding of one or more analytes to the microstructures can alter the charge carrying capability, in turn leading to changes in capacitance of electrode pairs, which can then be monitored, allowing analyte levels or concentrations to be derived. Binding of analytes can be achieved using a variety of techniques, including selection of mechanical properties of the microstructure, such as the presence of pores or other physical structures, the material from which the microstructures are manufactured, the use of coatings, or otherwise influencing the microstructure properties, such as by using magnetic microstructures.

Additionally, the microstructures and/or substrate can incorporate one or more materials or other additives, either within the body of the microstructure, or through addition of a coating containing the additive. The nature of the material or additive will vary depending on the preferred implementation and could include a bioactive material, a reagent for reacting with analytes in the subject, a binding agent for binding with analytes of interest, a material for binding one or more analytes of interest, a probe for selectively targeting analytes of interest, a material to reduce biofouling, a material to attract at least one substance to the microstructures, a material to repel or exclude at least one substance from the microstructures, a material to attract at least some analytes to the microstructures, or a material to repel or exclude analytes. In this regard, substances could include any one or more of cells, fluids, analytes, or the like. Example materials include polyethylene, polyethylene glycol, polyethylene oxide, zwitterions, peptides, hydrogels and self-assembled monolayers.

The material can be contained within the microstructures themselves, for example by impregnating the microstructures during manufacture, for example by introducing the material into the imprinting mold so that the material is applied to the microstructure surface during the molding process. Additionally and/or alternatively, the material can be incorporated into the resin prior to molding, or could be provided in a coating.

Accordingly, it will be appreciated that at least some of the microstructures can include a material for binding one or more analytes or interest, which can be used in order to target specific analytes of interest, allowing these to bind or otherwise attach to the microstructure, so that these can then be detected in situ using a suitable detection mechanism, such as by detecting changes in optical or electrical properties.

The analyte may be any compound, fluid or other substance that can be detected in the epidermis and/or dermis. In particular embodiments, the analyte is a marker of a condition, disease, disorder or a normal or pathologic process that occurs in a subject, or a compound which can be used to monitor levels of an administered substance in the subject, such as a medicament (e.g., drug, vaccine), an illicit substance (e.g. illicit drug), a non-illicit substance of abuse (e.g. alcohol or prescription drug taken for non-medical reasons), a poison or toxin, a chemical warfare agent (e.g. nerve agent, and the like) or a metabolite thereof. Suitable analytes include, but are not limited to a:
nucleic acid, including DNA and RNA, including short RNA species including microRNA, siRNA, snRNA, shRNA and the like:
antibody, or antigen-binding fragment thereof, allergen, antigen or adjuvant;
chemokine or cytokine;
hormone;
parasite, bacteria, virus, or virus-like particle, or a compound therefrom, such as a surface protein, an endotoxin, and the like;
epigenetic marker, such as the methylation state of DNA, or a chromatin modification of a specific gene/region;
peptide;
polysaccharide (glycan);
polypeptide;
protein; and
small molecule.

In particular embodiments, the analyte of interest is selected from the group consisting of a nucleic acid, antibody, peptide, polypeptide, protein and small molecule: especially a polypeptide and protein: most especially a protein.

In a further example, one or more microstructures include a treatment material, and wherein at least one treatment delivery mechanism is provided that controls release of the treatment material. In one preferred example, release of the treatment material is controlled by applying stimulation to the microstructure(s), for example by applying light, heat or electrical stimulation to release the treatment material.

In one preferred example, the treatment material is contained in a coating on the at least one microstructure and the stimulation is used to dissolve the coating on the microstructure and thereby deliver the treatment material. It will be appreciated that this technique can be applied to any treatment material that can be incorporated into a coating, and which can be selectively released using stimulation, such as mechanical, magnetic, thermal, electrical, electromagnetic or optical stimulation.

The nature of the treatment material will vary depending on the preferred implementation and/or the nature of the treatment being performed, including whether the treatment is cosmetic or therapeutic. Example treatment materials include, but are not limited to, nanoparticles, a nucleic acid, an antigen or allergen, parasites, bacteria, viruses, or virus-like particles, metals or metallic compounds, molecules, elements or compounds, DNA, protein, RNA, siRNA, sfRNA, iRNA, synthetic biological materials, polymers, drugs, or the like.

It will be appreciated that the use of coatings is not essential however, and additionally and/or alternatively treatment materials can be incorporated into the microstructures themselves.

Irrespective of how treatment materials are provided, the substrate can include a plurality of microstructures with different microstructures having different treatment materials and/or different treatment doses. In this case, the processing devices can control the therapy delivery mechanism to release treatment material from selected microstructures, thereby allowing different treatments to be administered, and/or allowing differential dosing, depending on the results of measurements performed on the subject. In particular, as will be described in more detail below, the processing devices typically perform an analysis at least in part using the measured response signals; and, use results of the analysis to control the at least one therapy delivery mechanism, thereby allowing personalized treatment to be administered substantially in real time.

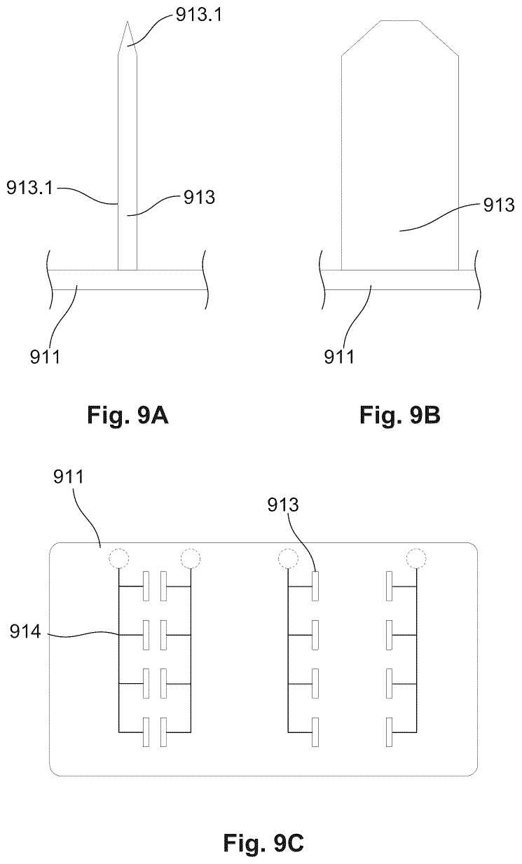

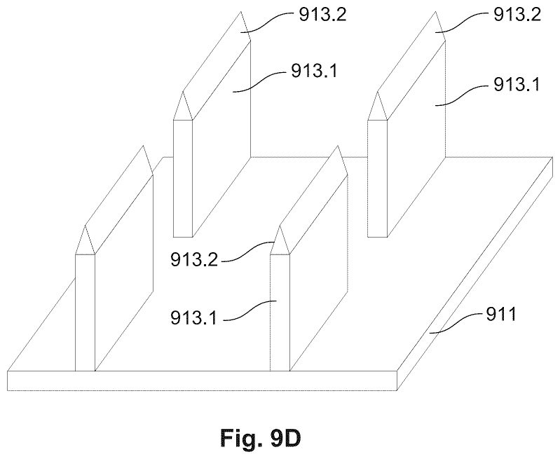

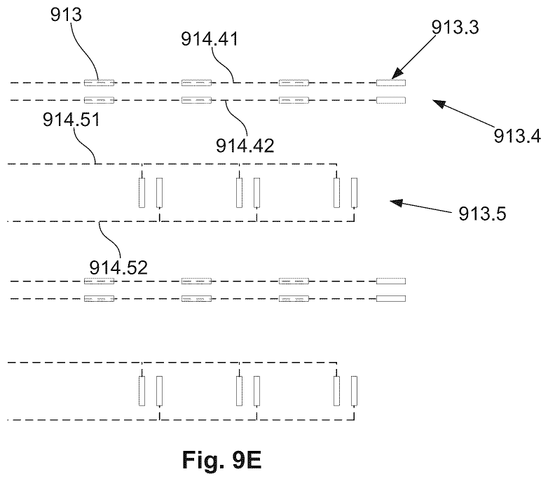

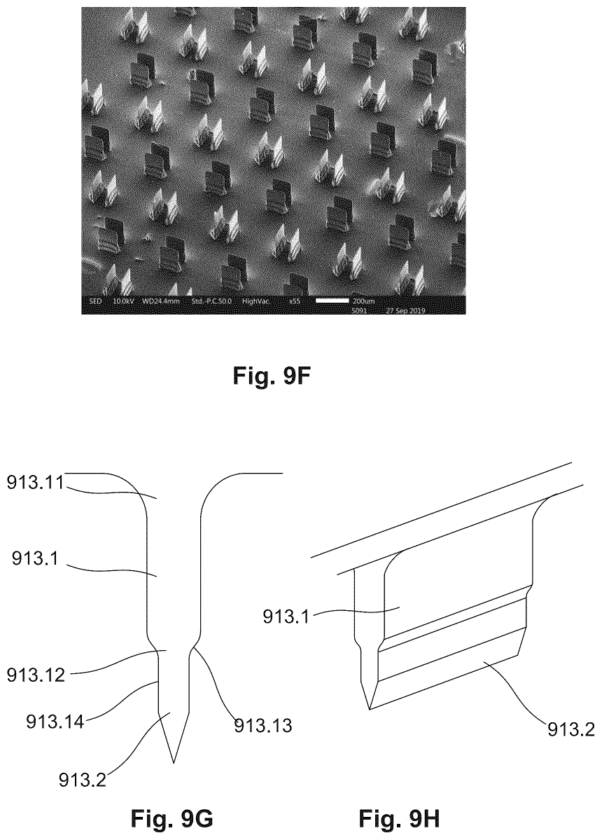

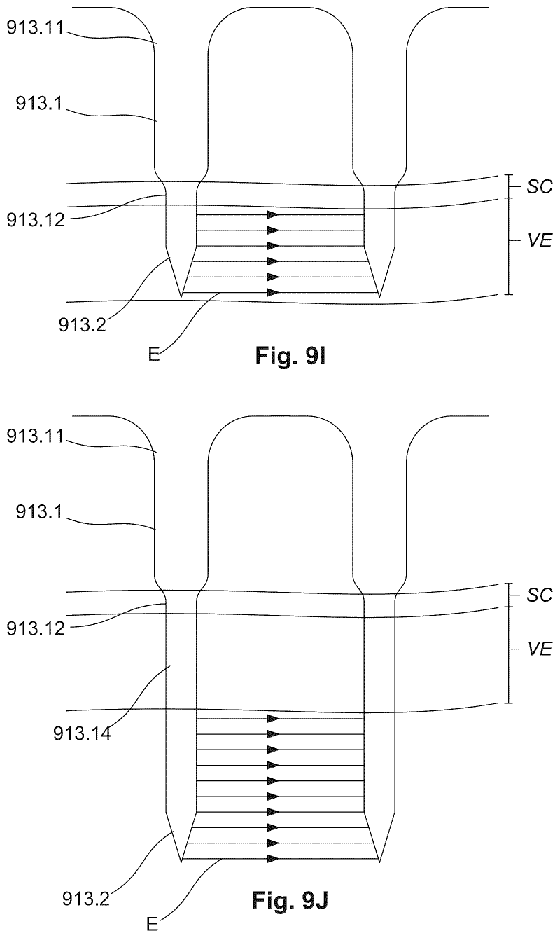

A specific example of a plate microstructure is shown is shown in FIGS. 9A to 9D.

In this example, the microstructure is a plate having a body 913.1 extending from the substrate 911, and a tip 913.2, which is tapered to facilitate penetration of the microstructure 913 into the stratum corneum. As shown in FIGS. 9C and 9D, different arrangements could be used but in general, pairs of microstructures are formed with the microstructures facing each other allowing signals to be applied between the microstructures or measured between the microstructures. Different separations between electrodes in pairs of electrodes can be used to allow different measurements to be performed and/or to alter the profile of stimulation of the tissue between the electrodes.

In the example of FIG. 9C, the pairs of microstructures are arranged in rows, with electrical connections 914 extending to each microstructure in the row. It will be appreciated that in practice this can be achieved by ensuring the microstructures in each row are electrically connected, with openings being provided between the rows to electrically isolate the rows.

A further example arrangement is shown in FIG. 9E, in which microstructures 913 are arranged in pairs 913.3, and with pairs arranged in offset rows, 913.4, 913.5. In this example, pairs in different rows are arranged orthogonally, so that the microstructures extend in different directions. This avoids all microstructures being aligned, which can in turn render a patch vulnerable to lateral slippage in a direction aligned with the microstructures. Additionally arranging the pairs orthogonally reduces interference, such as cross talk, between different pairs of electrodes, improving measurement accuracy and accounting for tissue anisotropy, particularly when measurements are being performed via multiple microstructure pairs simultaneously.

In one example, pairs of microstructures in each row can be provided with respective connections 914.41, 914.42:914.51, 914.52, allowing an entire row of microstructure pairs to be interrogated and/or stimulated simultaneously, whilst allowing different rows to be interrogated and/or stimulated independently.

A Scanning Electron Microscopy (SEM) image showing an array of pairs of offset plate microstructures is shown in FIG. 9F.

Specific examples of microstructures for performing measurements in the epidermis are shown in FIGS. 9G and 9H.

In this example, the microstructures are plates or blades, having a body 913.1, with a flared base 913.11, where the body joins the substrate, to enhance the strength of the microstructure. The body narrows at a waist 913.12 to define shoulders 913.13 and then extends to a tapered tip 913.2, in this example, via an untapered shaft 913.14. Typical dimensions are shown in Table 2 below.

TABLE 2

| Parameter | Min. | Typical | Max. | Units |
|---|---|---|---|---|
| Length | 50 | 150 | 300 | microns |
| Width | 50 | 150 | 300 | microns |
| Thickness | 10 | 25 | 50 | microns |
| Density | 100 | 600 | 5000 | cm⁻² |
| Tip radius | 0.1 | 1 | 5 | microns |
| Surface area per electrode | 2,000 | 22,500 | 200,000 | micron² |
| Buttress width at base | 30 | 75 | 150 | microns |

An example of a pair of the microstructures of on insertion into a subject is shown in FIG. 9I.

In this example, the microstructures are configured so that the tip 913.2 penetrates the stratum comeum SC and enters the viable epidermis VE. The waist 913.12, and in particular the shoulders 913.13 abut the stratum corneum SC so that the microstructure does not penetrate further into the subject, and so that the tip is prevented from entering the dermis. This helps avoid contact with nerves, which can lead to pain.

In this configuration, the body 913.1 of the microstructure can be coated with a layer of insulating material (not shown), with only the tip exposed. As a result a current signal applied between the microstructures, will generate an electric field E within the subject, and in particular within the viable epidermis VE, so that measurements reflect fluid levels in the viable epidermis VE.

However, it will be appreciated that other configurations can be used. For example, in the arrangement of FIG. 9J, the shaft 913.14 is lengthened so the tip 913.2 enters the dermis, allowing dermal (and optional epidermal) measurements to be performed.

In this example, typical dimensions are shown in Table 3 below.

TABLE 3

| Parameter | Min. | Typical | Max. | Units |
|---|---|---|---|---|
| Length | 50 | 250 | 450 | microns |
| Width | 50 | 250 | 450 | microns |
| Thickness | 10 | 30 | 50 | microns |
| Density | 100 | 600 | 5000 | cm⁻² |
| Tip radius | 0.1 | 1 | 5 | microns |
| Surface area per electrode | 10,000 | 62,500 | 427,000 | micron² |
| Buttress width at base | 30 | 75 | 150 | microns |

An example of the inter and intra pair spacing for these configurations are shown in Table 4 below.

TABLE 4

| Parameter | Min. | Typical | Max. | Units |
|---|---|---|---|---|
| Separation between microstructures | 10 | 100 | 1000 | microns |
| in a group or pair | | | | |
| Separation between groups of | 200 | 500 | 1000 | microns |
| microstructures | | | | |

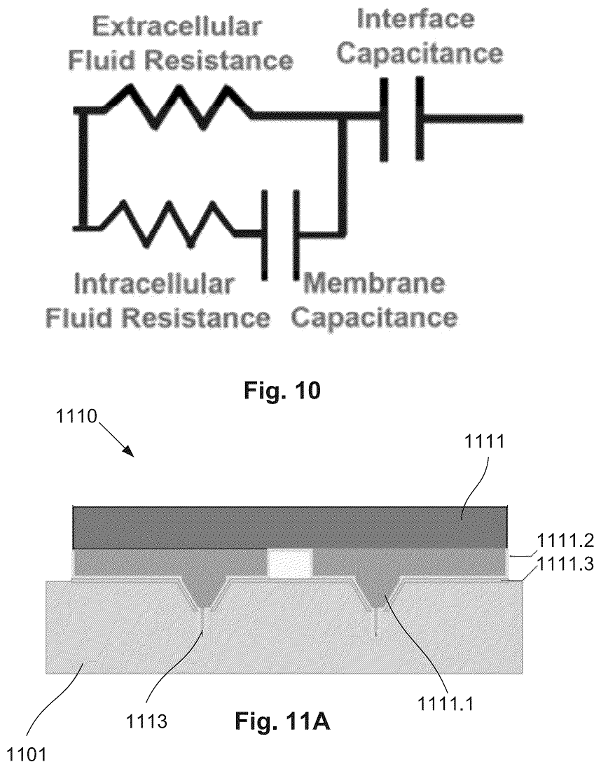

An example of a biophysical model that differentiates between the high and low frequency impedance response of tissue is shown in FIG. 10.

In this example, the model uses an equivalent circuit consisting of two parallel arms, including resistors and capacitors, which represent the low frequency Extracellular fluid response and the higher frequency intracellular fluid response, respectively. This model can also include further components to isolate confounding parameters, such as an interfacial capacitance, which represents the often large impedance dominating the lower frequencies of bio-impedance spectrums.

In this regard, the interfacial impedance or Electrode Polarization (EP) as it is better known, is a physical phenomenon that is fundamental to metal-electrolyte interactions, has been studied well for over a century and is present in two-electrode systems employed to measure the impedance response of an ionic environment. EP manifests as a double layer capacitor comprised of counter-ions adsorbed onto the surface of the electrode with a diffuse layer of ions surrounding it, driven by the source AC signal. It shields the larger response of the ionic environment from being measured at low frequencies. Above a certain frequency the effect of this capacitance is lowered. However with limited bandwidth of frequencies available in compact electronic packaging it can be noticeable. Since EP is a capacitance on a Bode plot of Phase vs. Frequency this effect shows up at frequencies where the phase is between −90 and −45 degrees. Preferably the EP effect is confined to as low a frequency regime as possible to ensure that a maximum of the frequency spectrum generated by the arrangements described herein is available for sensing changes in the ionic environment.

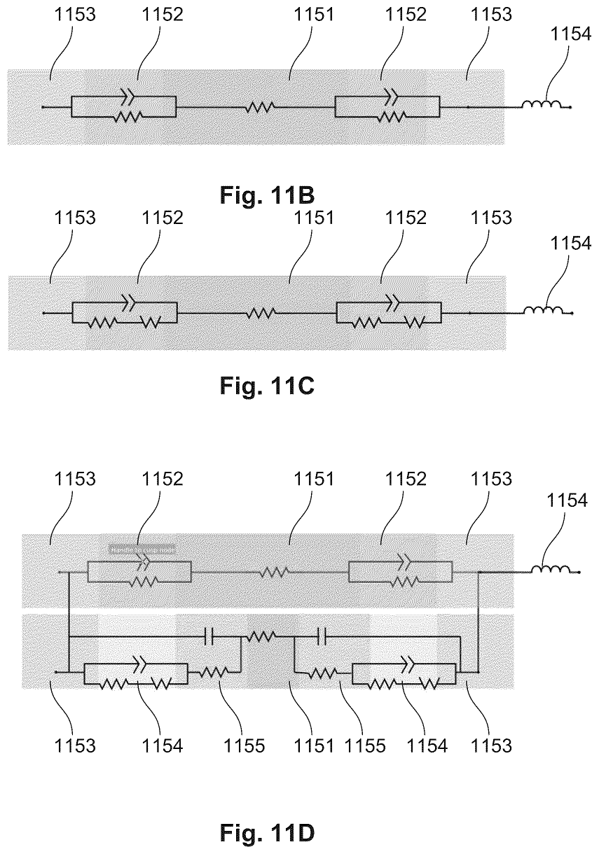

It will be appreciated that for more complex situations specific models can be developed, and a first example will now be described with reference to FIGS. 11A to 11D.

In this example, a testing scenario is shown, in which a patch 1110 is provided including a substrate 1111 having mesas 1111.1 projecting therefrom, which support microstructures 1113. In this example, the patch is coated with a conductive layer of gold 1111.2, allowing the microstructures to conduct electrical signals, with an additional layer of insulating material in the form of an insulating membrane being used to electrically insulate the gold material deposited on the surface of the substrate 1111. For testing, the patch is placed in contact with saline solution 1101, allowing impedance measurements of the saline to be captured.

In this example, a basic equivalent circuit model is as shown in FIG. 11B, with the model constructed to include components representing the saline 1151, an electrode saline interface 1152 and the electrode 1153, with a single inductor being used to represent impedance of the measuring device. In this example, the saline is represented by a single resistor, which the electrode saline interface 1152 is represented by a resistor and constant phase element (CPE) in parallel.

A more complex equivalent circuit model shown in FIG. 11C, replaces the resistor of the electrode saline interface 1152 with a resistor and Warburg impedance in series, which more accurately models ionic diffusion processes at the interface between the saline solution and the electrode.

A further more complex equivalent circuit model is shown in FIG. 11D. In this example, two branches are provided in parallel, with the first branch being equivalent to the equivalent circuit of FIG. 11B. A second branch is provided that models an electrode interface 1154 and interface between insulating membrane and saline 1155, with the former including a series connected resistor and Warburg impedance in parallel with a CPE, with this being connected in series to a resistor representing the interface between insulating membrane and saline 1155, which is in turn in parallel with a capacitor.

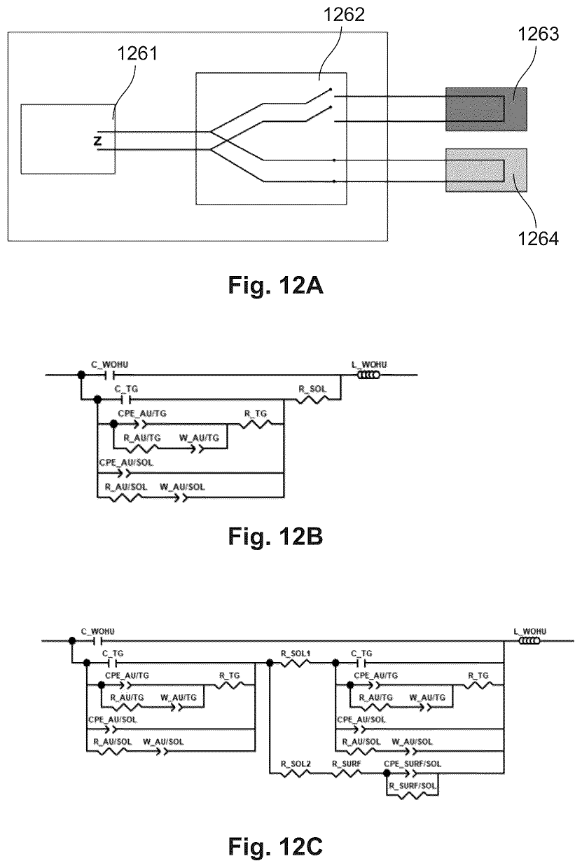

A dual sensing arrangement will now be described with reference to FIG. 12A.

In this example, a measuring device 1261 is connected via a multiplexer 1262, to a patch 1263 and a surface sensor 1264, which can be used to capture information regarding skin surface moisture, for example allowing sweat to be accounted for in measurements. An equivalent circuit for the liquid electrolyte model and associated electronics is shown in FIG. 12B, whilst a version also incorporating the surface sensor is shown in FIG. 12C, illustrating how more complex circuits can be constructed.

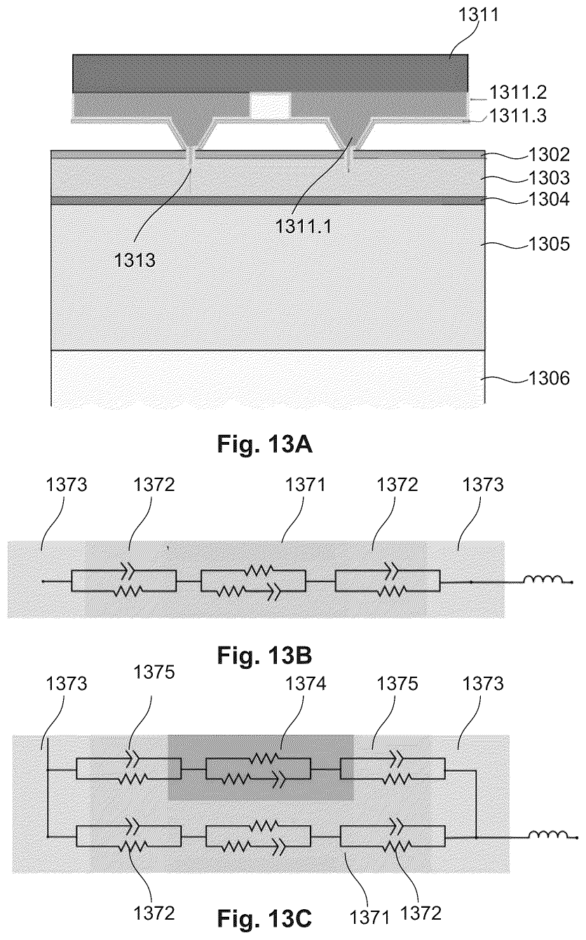

A further measurement example will now be described with reference to FIGS. 13A to 13C.

In this example, a patch 1310 is provided including a substrate 1311 having mesas 1311.1 projecting therefrom, which support microstructures 1313. In this example, the patch is again coated with a conductive gold layer 1311.2, and an additional layer of insulating membrane 1311.3. For measuring, the patching is inserted into the skin, penetrating the corneal layer 1302, and entering the viable epidermis 1303. In this example, the basal layer 1304, dermis 1305 and subcutaneous layer 1306 are not penetrated.

In this example, a basic equivalent circuit model is as shown in FIG. 13B, with the model constructed to include components representing the saline viable epidermis 1371, the electrode epidermis interface 1372 and the electrode 1373. Specifically, the epidermis is modelled as a series connected resistor and CPE in parallel with another resistor, whilst the interface is modelled as a resistor and CPE in parallel. A more complex equivalent circuit model is shown in FIG. 13C and includes a corneal layer and 1374 and associated interfaces 1375.

In another example, biophysical models can be constructed to represent the dependence of equivalent circuit component values, impedance values or similar electrical quantities on the physical, material and geometrical parameters of electrodes, and/or on biological quantities such as the volumes, concentrations or composition of fluids within living tissues.

Thus, it will be appreciated that models can be constructed for bioimpedance measurements, with the models being made to account for different factors influencing the resulting impedance measurements captured by the patch.

Persons skilled in the art will appreciate that numerous variations and modifications will become apparent. All such variations and modifications which become apparent to persons skilled in the art, should be considered to fall within the spirit and scope that the invention broadly appearing before described.

1. A system for analyzing measurements performed on a biological subject, the system including one or more processing devices configured to:
a) acquire subject data at least in part captured by a measurement system including:
i) at least one substrate including a plurality of microstructures configured to breach a functional barrier of the subject; and,
ii) a sensor operatively connected to at least one microstructure, the at least one sensor being configured to measure response signals from the at least one microstructure; and,
b) analyze the subject data using at least one model to determine an indicator at least partially indicative of a physiological state of the subject.

2. A system according to claim 1, wherein the subject data includes at least one of:
a) an identifier associated with at least one of:
i) the subject; and,
ii) a patch including the microstructures;
b) sensor data indicative of the measured response signals;
c) secondary sensor data indicative of measurements performed by one or more secondary sensors;
d) secondary sensor data indicative of measurements performed by one or more secondary sensors, wherein the secondary sensors include physiological sensors configured to sense one or more physiological parameters or signals;
e) subject trait data indicative of one or more subject traits;
f) subject parameters derived from previous measurements; and,
g) context data indicative of at least one of:
i) environmental parameters;
ii) environmental parameters measured by one or more environmental sensors; and,
iii) a subject context.

3. (canceled)

4. A system according to claim 1, wherein the one or more processing devices are configured to:
a) analyze the subject data to determine at least one metric; and,
b) apply the at least one metric to the model to determine the indicator, wherein the at least one model embodies a relationship between a physiological state and the at least one metric and wherein the at least one metric includes at least one of:
i) response signals;
ii) pre-processed response signals;
iii) values derived from the response signals;
iv) subject data;
v) an attribute of the subject data;
vi) a feature derived from an attribute of the subject data;
vii) an attribute of the context data;
viii) a feature from an attribute of the context data;
ix) an attribute statistically derived from measured response signal values including a least one of:
(1) a mean;
(2) a median;
(3) an average;
(4) a variance;
(5) a skew;
(6) a kurtosis;
(7) a percentile; and
(8) a cumulative distribution function.
x) a feature derived using at least one of:
(1) changes of attributes;
(2) rates of change of attributes;
(3) deviation of attributes from reference attributes;
(4) deviations of attributes from a baseline; and,
(5) one or more feature engineering algorithms.

5. (canceled)

6. (canceled)

7. (canceled)

8. (canceled)

9. A system according to claim 1, wherein at least one of:
a) the indicator is at least one of:
i) a predictive indicator;
ii) a classification;
iii) an index value; and,
iv) a measurement value; and,
b) wherein the one or more processing devices are configured to at least one of:
i) record the indicator;
ii) generate an output including at least one of:
(1) a representation of the indicator; and,
(2) a recommendation based on the indicator; and,
iii) cause an intervention to be performed based on the indicator.

10. (canceled)

11. A system according to claim 1, wherein at least one of:
a) the one or more processing devices are configured to process sensor data at least in part using at least one of:
i) blind source separation algorithms;
ii) independent component analysis; and,
iii) principal component analysis;
b) the one or more processing devices are configured to process the subject data by performing at least one of:
i) anomaly detection;
ii) data cleaning;
iii) bias correction;
iv) windowing;
v) normalization;
vi) standardization;
vii) base lining; and,
viii) signal processing; and,
c) the one or more processing devices are configured to:
i) perform anomaly detection by analyzing the subject data to identify at least one of:
(1) sudden changes in response signal values;
(2) outlier response signal changes;
(3) outlier response signal values; and,
(4) changes in response signal values corresponding to events; and,
ii) at least one of:
(1) perform data cleaning by at least one of:
(a) excluding anomalies from subsequent analysis;
(b) excluding sensor data including anomalies; and,
(c) performing anomaly correction; and,
(2) use pattern matching of anomalies to identify measurement device issues;
(3) analyze the anomalies; and,
(4) determine at least one metric using the anomaly; and,
d) the one or more processing devices are configured to perform bias correction based on at least one of:
i) individual senor characteristics;
ii) environmental parameters; and,
iii) physiological parameters.

12. (canceled)

13. A system according to claim 1, wherein the system includes one or more secondary sensors, and wherein the one or more processing devices are configured to at least one of:
a) synchronize the sensor data and secondary sensor data; and,
b) process the sensor data in accordance with secondary sensor data to at least one of:
i) perform bias correction;
ii) identify events; and,
iii) perform anomaly detection.

14. (canceled)

15. A system according to claim 1, wherein the one or more processing devices are configured to:
a) segment the subject data into a number of windows; and,
b) analyze the subject data using the windows and wherein at least one of:
i) the one or more processing devices are configured to analyze the data by analyzing at least one of:
(1) at least one metric for each window;
(2) a plurality of metrics for each window;
(3) a plurality of metrics for each of a plurality of windows;
(4) inter-window metric changes; and,
(5) intra-window metric changes.
ii) the one or more processing devices are configured to normalize subject data for each window;
iii) the one or more processing devices are configured to segment the subject data based on at least one of:
(1) fixed time intervals;
(2) events;
(3) events detected using secondary sensors; and,
(4) identified anomalies; and,
iv) the one or more processing devices are configured to:
(1) generate an indicator for each window; and,
(2) generate an indictor using metrics from multiple windows.

16. (canceled)

17. (canceled)

18. (canceled)

19. (canceled)

20. A system according to claim 1, wherein at least one of:
a) the one or more processing devices are configured to standardize the subject data to establish a baseline, the standardization being performed based on at least one of:
i) historical subject data; and,
ii) physiological parameters;
b) the one or more processing devices are configured to perform baselining by using a baseline to at least one of:
i) adjust subject data;
ii) flag subject data of interest;
c) the one or more processing devices are configured to perform baselining by at least one of:
i) comparison to a baseline; and,
ii) using a baselining computational model.

21. (canceled)

22. (canceled)

23. A system according to claim 1, wherein the at least one model is at least one of:
a) a biophysical model;
b) a computational model;
c) a statistical model;
d) a biochemical model;
e) obtained using reference metrics derived from subject data measured for one or more referenced subjects having known physiological states;
f) obtained and/or fit using at least one of machine learning and statistical inference; and,
g) obtained and/or fit using at least one of:
i) linear or non-linear regression;
ii) logistic regression;
iii) clustering algorithms;
iv) neural networks;
v) random forests;
vi) decision trees;
vii) Bayesian algorithms;
viii) Random effects, fixed effects or mixed effects modelling;
ix) Random field modelling;
x) gaussian processes; and,
xi) ensemble methods.

24. (canceled)

25. (canceled)

26. (canceled)

27. A system according to claim 1, wherein at least one of:
a) the one or more electronic devices are configured to determine an indicator by performing at least one of:
i) pattern matching;
ii) a longitudinal analysis; and,
iii) comparison to a threshold;
b) the one or more processing devices are configured to determine a physiological state indicator indicative of at least one of:
i) a predicted physiological state of the subject;
ii) a presence, absence or degree of a medical condition;
iii) a prognosis associated with a medical condition;
iv) a presence, absence, level or concentration of a biomarker;
v) a presence, absence, level or concentration of an analyte;
vi) fluid levels in the subject;
vii) blood oxygenation; and,
viii) bioelectric activity.

28. (canceled)

29. A system according to claim 1, wherein at least one of:
a) the measurement system includes a signal generator operatively connected to at least one microstructure to apply a stimulatory signal to the at least one microstructure; and,
b) the response signals or stimulatory signals are at least one of:
i) mechanical;
ii) magnetic;
iii) thermal;
iv) electrical;
v) electromagnetic; and
vi) optical.

30. (canceled)

31. A system according to claim 1, wherein the measurement system includes:
a) a patch including the substrate and microstructures; and,
b) a monitoring device that is configured to at least one of:
i) perform the measurements;
ii) generate the subject data;
iii) provide the subject data to the one or more processing devices; and,
iv) display an output based on the indicator.

32. A system according to claim 31, wherein at least one of:
a) the monitoring device is at least one of:
i) inductively coupled to the patch;
ii) attached to the patch; and,
iii) placed in contact with the patch at least one of:
(1) when measurements are to be performed; and,
(2) when sensor data is retrieved from the sensor; and,
b) the sensor is at least one of:
i) mounted on the patch; and,
ii) provided in the monitoring device; and,
c) the system includes one or more secondary sensors, and wherein the secondary sensors are at least one of:
i) mounted on a patch;
ii) provided in the monitoring device; and,
iii) in communication with the monitoring device.

33. (canceled)

34. (canceled)

35. A system according to claim 1, wherein at least one of:
a) at least some of the microstructures include at least one electrode that at least one of:
i) extends over a length of a distal portion of the microstructure;
ii) extends over a length of a portion of the microstructure spaced from the tip;
iii) is positioned proximate a distal end of the microstructure;
iv) is positioned proximate a tip of the microstructure;
v) extends over at least 25% of a length of the microstructure;
vi) extends over less than 50% of a length of the microstructure;
vii) extends over about 60 μm of the microstructure; and,
viii) is configured to be positioned in a viable epidermis of the subject in use;
b) the substrate includes electrical connections to allow electrical signals to be applied to and/or received from respective microstructures;
c) at least some of the microstructures include an insulating layer extending over at least one of:
i) part of a surface of the microstructure;
ii) a proximal end of the microstructure;
iii) at least half of a length of the microstructure;
iv) about 90 μm of the microstructure; and,
v) at least part of a tip portion of the microstructure.

36. (canceled)

37. (canceled)

38. A method according to claim 1, wherein at least one of:
a) the microstructures include at least one of:
i) plate microstructures;
ii) at least partially tapered plate microstructures;
iii) plate microstructures having a substantially rounded rectangular cross sectional shape;
iv) spaced apart substantially parallel plate microstructures;
v) spaced apart rows of microstructures;
vi) pairs of spaced apart microstructures; and,
vii) groups of microstructures; and,
b) at least one of:
i) at least some microstructures are angularly offset;
ii) at least some microstructures are orthogonally arranged;
iii) adjacent pairs of microstructures are orthogonally arranged;
iv) adjacent pairs of microstructures are angularly offset;
v) pairs of microstructures are arranged in rows, and the pairs of microstructures in one row are orthogonally arranged relative to pairs of microstructures in other rows; and,
vi) pairs of microstructures are arranged in rows, and the pairs of microstructures in one row are angularly offset relative to pairs of microstructures in other rows.

39. (canceled)

40. A system according to claim 1, wherein at least one of:
a) the microstructures have a spacing that is at least one of:
i) less than 1 mm;
ii) about 0.5 mm;
iii) about 0.2 mm;
iv) about 0.1 mm; and,
v) more than 10 μm;
b) at least some of the microstructures have at least one of:
i) a length that is at least one of:
(1) less than 300 μm;
(2) about 150 μm;
(3) greater than 100 μm; and,
(4) greater than 50 μm;
ii) a maximum width that is at least one of:
(1) greater than the length;
(2) about the same as the length;
(3) less than 300 μm;
(4) about 150 μm; and,
(5) greater than 50 μm; and,
iii) a thickness that is at least one of:
(1) less than 50 μm;
(2) about 25 μm; and,
(3) greater than 10 μm;
c) at least some of the microstructures have a tip that at least one of:
i) has a length that is at least one of:
(1) less than 50% of a length of the microstructure;
(2) at least 10% of a length of the microstructure; and,
(3) about 30% of a length of the microstructure; and,
ii) has a sharpness of at least one of:
(1) at least 0.1 μm;
(2) less than 5 μm; and,
(3) about 1 μm;
d) the microstructures have a density that is at least one of:
i) less than 5000 per cm2;
ii) greater than 100 per cm2; and,
iii) about 600 per cm2;
e) at least some microstructures include an electrode having a surface area of at least one of:
i) less than 200,000 μm2;
ii) about 22,500 μm2; and,
iii) at least 2,000 μm2;
f) the microstructures include anchor microstructures used to anchor the substrate to the subject and wherein the anchor microstructures at least one of:
i) include anchoring structures;
ii) have a length greater than that of other microstructures; and,
iii) enter the dermis.

41. (canceled)

42. (canceled)

43. (canceled)

44. (canceled)

45. (canceled)

46. A system according to claim 1, wherein the microstructures include a material including at least one of:
a) a bioactive material;
b) a reagent for reacting with analytes in the subject;
c) a binding agent for binding with analytes of interest;
d) a probe for selectively targeting analytes of interest;
e) a material to reduce biofouling;
f) a material to attract at least one substance to the microstructures;
g) a material to repel at least one substance from the microstructures;
h) a material to attract at least some analytes to the projections; and,
i) a material to repel at least some analytes from the projections.

47. A system according to claim 1, wherein at least some of the microstructures are coated with a coating that at least one of:
a) modifies surface properties to at least one of:
i) increase hydrophilicity;
ii) increase hydrophobicity; and,
iii) minimize biofouling;
b) attracts at least one substance to the microstructures;
c) repels at least one substance from the microstructures;
d) acts as a barrier to preclude at least one substance from the microstructures; and,
e) includes at least one of:
i) polyethylene;
ii) polyethylene glycol;
iii) polyethylene oxide;
iv) zwitterions;
v) peptides;
vi) hydrogels; and,
vii) SAMs.

48. A method for analyzing measurements performed on a biological subject, the method including, in one or more processing devices:
a) acquiring subject data at least in part captured by a measurement system including:
i) at least one substrate including a plurality of microstructures configured to breach a functional barrier of the subject; and,
ii) a sensor operatively connected to at least one microstructure, the at least one sensor being configured to measure response signals from the at least one microstructure; and,
b) analyzing the subject data using at least one model to determine an indicator at least partially indicative of a physiological state of the subject.
